SEQUENCE LISTING (1 sequence)

SEQ ID NO: 1 - DNA, 78 bp, synthetic construct
  source 1..78: mol_type = other DNA
cactccctctctgcgcgctcgctcgctcactgaggccgggcgaccaaaggtcgcccacgcccgggctttgcccgggcg

(19) United States
(12) Patent Application Publication
(10) Pub. No.: US 2023/0405014 A1
(43) Pub. Date: Dec. 21, 2023

(54) USE OF AN IRAK4 MODULATOR FOR GENE THERAPY

(71) Applicant: Genzyme Corporation, Cambridge, MA (US)

(72) Inventors: Sourav Roy CHOUDHURY, Waban, MA (US); Mona MOTWANI, Framingham, MA (US); Christian MUELLER, Concord, MA (US); John REED, Miami Beach, FL (US)

(21) Appl. No.: 18/299,640

(22) Filed: Apr. 12, 2023

Related U.S. Application Data
(60) Provisional application No. 63/330,245, filed on Apr. 12, 2022.

(51) Int. Cl. A61K 31/4162; C12N 15/86; C07K 14/015; A61K 48/00; C12N 5/0786

(52) U.S. Cl. CPC A61K 31/4162; C12N 15/86; C07K 14/015; A61K 48/005; A61K 48/0075; C12N 5/0645; C12N 2750/14143; C12N 2502/1121

(57) ABSTRACT

Provided herein are methods for enhancing gene therapy in an individual by administering an IRAK degrader with the gene therapy to suppress innate immunity to the gene therapy. In some embodiments, the gene therapy uses an adeno-associated virus (AAV) vector, an adenovirus vector, a lentivirus vector, a Herpes simplex virus (HSV) vector or a lipid nanoparticle. Also provided herein are methods for selecting an individual for treatment with an IRAK degrader in combination with a gene therapy agent.

### CROSS REFERENCE TO RELATED APPLICATIONS

This application claims the priority benefit of U.S. Provisional Application No. 63/330,245, filed Apr. 12, 2022, which is incorporated by reference in its entirety

### REFERENCE TO AN ELECTRONIC SEQUENCE LISTING

The contents of the electronic sequence listing (159792018200SEQLIST.xml; Size: 1,990 bytes; and Date of Creation: Apr. 12, 2023) is herein incorporated by reference in its entirety.

### FIELD OF THE INVENTION

The present invention relates to methods for enhancing gene therapy in an individual by administering an IRAK degrader with the gene therapy to suppress innate immunity to the gene therapy. In some aspects, the invention provides methods for selecting an individual for treatment with an IRAK degrader in combination with a gene therapy agent.

### BACKGROUND OF THE INVENTION

Success of gene therapy for treatment of rare genetic diseases relies heavily on Adeno-associated virus (AAV) viral vectors that provide many attractive features including tissue specific tropism, transduction of quiescent cells and maintenance of modified gene expression. However, immune responses to AAV vectors pose a major challenge for successful clinical translation. The capsid, viral genome as well as transgene trigger immune responses that involve activation of both innate and adaptive arms of immune system. The innate immune system activated by the TLR pathway subsequently evokes an adaptive immune response in B and T cells in humans exposed to pathogens (Iawaski, A and Medzhitov, R, Nat Immunol 2004 5(10):987-985). Based on several mouse studies it is shown that the endosomal DNA sensor TLR9 that recognizes CpG rich hypomethylated DNA plays an important role in the genome recognition of AAV vectors. TLR9 triggered signaling cascade activates adaptive immune responses that ultimately lead to clearance of transgene induced cells by T cell mediated cell cytotoxicity. Mice lacking TLR9 sensor show longer maintenance of transgene expression and diminished immune response (Ashley, S N et al., Cell Immunol 2019 December; 346:103997, and Faust S M et al., J Clin Invest 2013 123(7):2994-3001). In line with this data form hemophilia clinical trials reveal that reduction in total number of CpG bases reduces the need for pharmacological immunosuppressants and show reduced cytotoxic T lymphocyte responses in hemophilia patients whereas patients who received vectors that contained more CpG bases in their transgene had much larger requirement for immune suppressants (Wright, JF Mol. Ther. 2020 28(3):701-703). While broadly acting immune suppressants have been successful in improving AAV delivery in clinical trials, they still result in loss of transgene expression and present with side effects and risk of opportunistic infections. Developing vectors with no CpG bases is a challenge because non-codon optimized vectors (i.e. vectors with no or low CpG content) show poor transgene expression (Wright, JF Mol. Ther. 2020 28(3):701-703). Hence, what is needed is a different class of immune degraders that show enhanced specificity and low side effects.

All references cited herein, including patent applications and publications, are incorporated by reference in their entirety.

### BRIEF SUMMARY OF THE INVENTION

In some aspects, the invention provides methods for delivering nucleic acid to a cell of an individual, the method comprising a) administering an IRAK degrader to the individual, and b) administering a gene therapy agent to the individual. In some aspects, the invention provides methods for treating an individual in need thereof with a gene therapy agent, the method comprising a) administering an IRAK degrader to the individual, and b) administering the gene therapy agent to the individual. In some aspects, the invention provides methods for improving gene therapy in an individual, the method comprising a) administering an IRAK degrader to the individual, and b) administering a gene therapy agent to the individual. In some aspects, the invention provides methods for suppressing an immune response to a gene therapy agent, the method comprising a) administering an IRAK degrader to the individual, and b) administering a gene therapy agent to the individual.

In some embodiments of the invention, the IRAK degrader modulates the activity or expression of an IRAK protein kinase. In some embodiments, the IRAK protein kinase is an IRAK-1 protein kinase, an IRAK-2 protein kinase, an IRAK-3 protein kinase protein kinase, or an IRAK-4 protein kinase. In some embodiments, the IRAK degrader modulates the activity or expression of an IRAK-4 protein kinase. In some embodiments, the IRAK degrader comprises the compound of formula [I]:

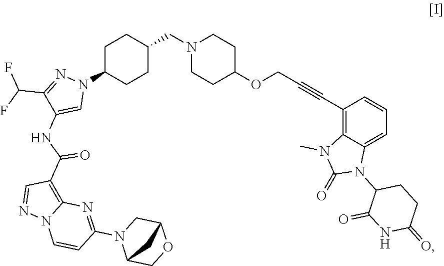 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21]

or a pharmaceutically acceptable salt thereof.

In some embodiments, the gene therapy agent comprises a viral vector. In some embodiments, the viral vector is an AAV particle. In some embodiments, the AAV particle comprises an AAV1 capsid, an AAV2 capsid, an AAV3 capsid, an AAV4 capsid, an AAV5 capsid, an AAV6 capsid, an AAV7 capsid, an AAV8 capsid, an AAVrh8 capsid, an AAV9 capsid, an AAV10 capsid, an AAVrh10 capsid, an AAV11 capsid, an AAV12 capsid, an AAVrh32.33 capsid, An AAV-XL32 capsid, an AAV-XL32.1 capsid, an AAV LK03 capsid, an AAV2R471A capsid, an AAV2/2-7m8 capsid, an AAV DJ capsid, an AAV DJ8 capsid, an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, a mouse AAV capsid rAAV2/HBoV1 (chimeric AAV/human bocavirus virus 1), an AAV2HBKO capsid, an AAVPHP.B capsid or an AAVPHP.eB capsid, or a functional variant thereof. In some embodiments, the AAV capsid comprises a tyrosine mutation, a heparin binding mutation, or an HBKO mutation. In some embodiments, the AAV viral particle comprises an AAV genome comprising one or more inverted terminal repeats (ITRs), wherein the one or more ITRs is an AAV1 ITR, an AAV2 ITR, an AAV3 ITR, an AAV4 ITR, an AAV5 ITR, an AAV6 ITR, an AAV7 ITR, an AAV8 ITR, an AAVrh8 ITR, an AAV9 ITR, an AAV10 ITR, an AAVrh10 ITR, an AAV11 ITR, or an AAV12 ITR. In some embodiments, the one or more ITRs and the capsid of the AAV particle are derived from the same AAV serotype. In some embodiments, the one or more ITRs and the capsid of the AAV particles are derived from different AAV serotypes.

In some embodiments, the viral vector is an adenoviral particle. In some embodiments, the adenoviral particle comprises a capsid from Adenovirus serotype 2, 1, 5, 6, 19, 3, 11, 7, 14, 16, 21, 12, 18, 31, 8, 9, 10, 13, 15, 17, 19, 20, 22, 23, 24-30, 37, 40, 41, AdHu2, AdHu 3, AdHu4, AdHu24, AdHu26, AdHu34, AdHu35, AdHu36, AdHu37, AdHu41, AdHu48, AdHu49, AdHu50, AdC6, AdC7, AdC69, bovine Ad type 3, canine Ad type 2, ovine Ad, or porcine Ad type 3, or a functional variant thereof.

In some embodiments, the viral vector is a lentiviral particle. In some embodiments, the lentiviral particle is pseudotyped with vesicular stomatitis virus (VSV), lymphocytic choriomeningitis virus (LCMV), Ross river virus (RRV), Ebola virus, Marburg virus, Mokala virus, Rabies virus, RD114, or a functional variant thereof.

In some embodiments, the viral vector is a Herpes simplex virus (HSV) particle. In some embodiments, the HSV particle is an HSV-1 particle or an HSV-2 particle, or a functional variant thereof.

In some embodiments, the gene therapy agent comprises a lipid nanoparticle.

In some embodiments, the gene therapy agent comprises nucleic acid encoding a heterologous transgene. In some embodiments, the heterologous transgene is operably linked to a promoter. In some embodiments, the promoter is a constitutive promoter, a tissue-specific promoter, or an inducible promoter.

In some embodiments, the IRAK degrader is administered before, at the same time, or after administration of the gene therapy agent. In some embodiments, the individual has a disease or disorder suitable for treatment by gene therapy. In some embodiments, the disease or disorder is a monogenic disease or disorder. In some embodiments, the gene therapy agent is administered intravenously, intraperitoneally, intra-arterially, intramuscularly, subcutaneously, or intrahepatically. In some embodiments, the IRAK degrader is administered orally, intravenously, intraperitoneally, intra-arterially, intramuscularly, subcutaneously, or intrahepatically.

In some aspects, the invention provides methods for delivering a nucleic acid to a cell of an individual, the method comprising a) incubating innate immune cells from the individual with the gene therapy agent, b) analyzing the innate immune cells for the expression of one or more cytokines wherein expression of a cytokine signature following incubation with the gene therapy agent identifies an individual with innate immunity to the gene therapy agent, c) administering an IRAK degrader to the individual identified in step b), and d) administering the gene therapy agent to the individual identified in step b). In some aspects, the invention provides methods for treating an individual in need thereof, the method comprising a) incubating innate immune cells from the individual with the gene therapy agent, b) analyzing the innate immune cells for the expression of one or more cytokines wherein expression of a cytokine signature following incubation with the gene therapy agent identifies an individual with innate immunity to the gene therapy agent, c) administering an IRAK degrader to the individual identified in step b), and d) administering the gene therapy agent to the individual identified in step b). In some aspects, the invention provides methods for selecting an individual for treatment with a gene therapy agent and an IRAK degrader, the method comprising a) incubating innate immune cells from the individual with the gene therapy agent, b) analyzing the innate immune cells for the expression of one or more cytokines wherein expression of a cytokine signature following incubation with the gene therapy agent identifies an individual for treatment with a gene therapy agent and an IRAK degrader, c) selecting the individual identified in step b) for treatment with a gene therapy agent and an IRAK degrader.

In some embodiments, the innate immune cell is a dendritic cell, a monocyte, a macrophage or a natural killer (NK) cell. In some embodiments, the innate immune cells are isolated from peripheral blood mononuclear cells from the individual. In some embodiments, the innate immune cell is a dendritic cell. In some embodiments, the dendritic cell is derived from a monocyte of the individual.

In some embodiments, the method further comprises isolating monocytes from the individual and incubating the monocytes in dendritic cell culture media to derive dendritic cells from the monocytes prior to incubating the dendritic cells with the gene therapy agent. In some embodiments, the monocytes are CD14+ monocytes. In some embodiments, the monocytes are incubates with the dendritic cell culture media for about 5 to about 10 days or about 7 to about 8 days to derive dendritic cells from the monocytes. In some embodiments, the dendritic cells are replated prior to the incubation with the gene therapy agent of step c). In some embodiments, the dendritic cells are replated into microwell dishes.

In some embodiments, the gene therapy agent is a viral vector, and wherein the innate immune cells are incubated with the viral vector at an MOI of about 1×10³ to about 1×10⁵ or about 1×104. In some embodiments, the gene therapy agent is a non-viral vector, and wherein the innate immune cells are incubated with the non-viral vector at a concentration of about 1 ng/mL to about 1 mg/mL. In some embodiments, the innate immune cells are incubated with the gene therapy agent for about 12 hours to about 36 hours or about 24 hours.

In some embodiments, the cytokine signature comprises increased expression of one or more of IL6, TNFα, IL-1β, MCP1 and MIP-1α. In some embodiments, the cytokine signature comprises increased expression of IL6, TNFα, IL-1β, MCP1 and MIP-1α. In some embodiments, the cytokine signature comprises increased expression of IL6, TNFα, and IL-1β. In some embodiments, expression of the cytokines in the cytokine signature is increased compared to a suitable control. In some embodiments, the suitable control is the expression of the cytokines in the cytokine signature from innate immune cells that are not incubated with the gene therapy agent or wherein the suitable control is expression of the cytokines in the cytokine signature from innate immune cells prior to incubation with the gene therapy agent.

In some aspects, the invention provides uses of a composition in the manufacture of a medicament for delivering nucleic acid to a cell of an individual in need thereof, wherein the composition comprises a gene therapy agent, and wherein the composition is formulated for use in combination with an IRAK degrader. In some aspects, the invention provides uses of a composition in the manufacture of a medicament for delivering nucleic acid to a cell of an individual in need thereof, wherein the composition comprises an IRAK degrader, and wherein the composition is formulated for use in combination with a gene therapy agent. In some aspects, the invention provides uses of a composition in the manufacture of a medicament for treating an individual in need of gene therapy, wherein the composition comprises a gene therapy agent, and wherein the composition is formulated for use in combination with an IRAK degrader. In some aspects, the invention provides uses of a composition in the manufacture of a medicament for treating an individual in need of gene therapy, wherein the composition comprises an IRAK degrader, and wherein the composition is formulated for use in combination with a gene therapy agent. In some aspects, the invention provides uses of a composition in the manufacture of a medicament for modulating an immune response to gene therapy in an individual in need of gene therapy, wherein the composition comprises a gene therapy agent, and wherein the composition is formulated for use in combination with an IRAK degrader. In some aspects, the invention provides uses of a composition in the manufacture of a medicament for modulating an immune response to gene therapy in an individual, wherein the composition comprises an IRAK degrader, and wherein the composition is formulated for use in combination with a gene therapy agent. In some embodiments, the gene therapy agent is an AAV particle, an adenovirus particle, a lentivirus particle, a HSV particle, or a lipid nanoparticle. In some embodiments, the IRAK degrader is an IRAK-4 degrader.

In some aspects, the invention provides compositions comprising a gene therapy agent for use in delivering nucleic acid to a cell of an individual in need thereof, wherein the gene therapy agent is used in combination with an IRAK degrader. In some aspects, the invention provides compositions comprising an IRAK degrader for use in delivering nucleic acid to a cell of an individual in need thereof, wherein the IRAK degrader is used in combination with a gene therapy agent. In some aspects, the invention provides compositions comprising a gene therapy agent for use in treating an individual in need of gene therapy, wherein the gene therapy agent is used in combination with an IRAK degrader. In some aspects, the invention provides compositions comprising an IRAK degrader for use in treating an individual in need of gene therapy, wherein the IRAK degrader is used in combination with a gene therapy agent. In some aspects, the invention provides compositions comprising an IRAK degrader for modulating an immune response to gene therapy in an individual in need of gene therapy, wherein the IRAK degrader is used in combination with a gene therapy agent. In some aspects, the invention provides compositions comprising an IRAK degrader for suppressing an immune response to gene therapy in an individual in need of gene therapy, wherein the IRAK degrader is used in combination with a gene therapy agent. In some embodiments, the gene therapy agent is an AAV particle, an adenovirus particle, a lentivirus particle, a HSV particle, or a lipid nanoparticle. In some embodiments, the IRAK degrader is an IRAK-4 degrader.

In some embodiments, the invention provides kits for use in the methods of the invention.

### BRIEF DESCRIPTION OF FIGURES

FIG. 1 shows the general schema to assess effect of the IRAK4 degraders KT-474 on human dendritic cells treated with AAVs.

FIGS. 2A-2C show that co-treatment of AAV with KT-474 results in dampening of cytokine release. FIG. 2A shows release of cytokine IL1b induced by AAV administered by itself or with various concentration of KT-474. FIG. 2B shows release of cytokine IL6 induced by AAV administered by itself or with various concentration of KT-474. FIG. 2C shows release of cytokine TNFA induced by AAV administered by itself or with various concentration of KT-474.

FIG. 3 shows that treatment with KT-474 causes no cellular toxicity in primary human monocytic dendritic cells.

FIG. 4 shows that treatment with KT-474 causes degradation of IRAK4 protein in primary human monocytic dendritic cells

FIG. 5. shows a schematic of an in vivo mouse experiment used to assess the effect of the IRAK4 degrader KT-474 on the immune response induced by AAV.

FIGS. 6A-6B show that treatment with KT-474 reduces transgene LacZ specific CD8 T cells. FIG. 6A shows reduction of transgene LacZ specific CD8 T cells in PBMCs derived from peripheral blood on 14 days post AAV injections. FIG. 6B shows reduction of transgene LacZ specific CD8 T cells in spleen on 21 days post AAV injections

FIG. 7 shows a general schematic of an in vivo mouse model to assess the effect of oral administration on IRAK4 degrader KT-474 on the immune response induced by AAV.

### DETAILED DESCRIPTION

In some aspects, the invention provides methods for delivering nucleic acid to a cell of an individual, the method comprising a) administering an IRAK degrader (e.g., an IRAK-4 degrader to the individual, and b) administering a gene therapy agent comprising nucleic acid. As used herein, a “gene therapy agent” can be the therapeutic component and/or the carrier component of the gene therapy that is to be administered (e.g., a nucleic acid (e.g., a close ended DNA), a viral vector (e.g., an AAV vector, an adenovirus vector, a lentivirus vector, a HSV vector), a lipid nanoparticle, all or a portion of an antibody (e.g., nanobody, Fc region, others) to the individual. In some aspects, the invention provides methods for treating an individual in need thereof with a composition comprising a gene therapy agent, the method comprising a) administering an IRAK degrader to the individual, and b) administering a gene therapy agent to the individual. In some aspects, the invention provides methods for improving gene therapy in an individual, the method comprising a) administering an IRAK degrader to the individual, and b) administering a gene therapy agent to the individual. In some aspects, the invention provides methods for modulating an immune response to a gene therapy agent, the method comprising a) administering an IRAK degrader to the individual, and b) administering a gene therapy agent to the individual. In some aspects, the invention provides methods for suppressing an immune response to a gene therapy agent, the method comprising a) administering an IRAK degrader to the individual, and b) administering a gene therapy agent to the individual. In some aspects, the invention provides methods for inducing tolerance to a gene therapy agent, the method comprising a) administering an IRAK degrader to the individual, and b) administering a gene therapy agent to the individual.

In some aspects, the invention provides methods for delivering a nucleic acid to a cell of an individual, the method comprising a) incubating the innate immune cells from the individual with the gene therapy agent (e.g., an AAV particle, an adenovirus particle, a lentivirus particle, a HSV particle, or a lipid nanoparticle), b) analyzing the innate immune cells for the expression of one or more cytokines wherein expression of a cytokine signature following incubation with the gene therapy agent identifies an individual with immunity (e.g., innate immunity, adaptive immunity) to the gene therapy agent, c) administering an IRAK degrader (e.g., an IRAK-4 degrader) to the individual identified in step b), and d) administering the gene therapy agent to the individual identified in step b). In some embodiments, the innate immune cells are dendritic cells, monocytes, macrophages, or natural killer (NK) cells. In some embodiments, the method further includes the steps of isolating innate immune cells from the individual prior to incubating the innate immune cells with the gene therapy agent. In some embodiments, the method further includes the steps of isolating monocytes from the individual and, incubating the monocytes in dendritic cell culture media to derive dendritic cells from the monocytes prior to incubating the dendritic cells with the gene therapy agent. As used herein, the term to “derive” dendritic cells includes differentiation of cells (e.g., monocytes) to produce dendritic cells.

In some aspects, the invention provides methods for treating an individual in need thereof, the method comprising a) incubating the innate immune cells from the individual with the gene therapy agent (e.g., an AAV particle, an adenovirus particle, a lentivirus particle, a HSV particle, or a lipid nanoparticle), b) analyzing the innate immune cells for the expression of one or more cytokines wherein expression of a cytokine signature following incubation with the gene therapy agent identifies an individual with immunity (e.g., innate immunity, adaptive immunity) to the gene therapy agent, c) administering an IRAK degrader (e.g., an IRAK-4 degrader to the individual identified in step b), and d) administering the gene therapy agent to the individual identified in step b). In some embodiments, the innate immune cells are dendritic cells, monocytes, macrophages, or natural killer (NK) cells. In some embodiments, the method further includes the steps of isolating innate immune cells from the individual prior to incubating the innate immune cells with the gene therapy agent. In some embodiments, the method further includes the steps of isolating monocytes from the individual and, incubating the monocytes in dendritic cell culture media to derive dendritic cells from the monocytes prior to incubating the dendritic cells with the gene therapy agent.

In some aspects, the invention provides methods for selecting an individual for treatment with a gene therapy agent (e.g., an AAV particle, an adenovirus particle, a lentivirus particle, a HSV particle, or a lipid nanoparticle) and an IRAK degrader (e.g., an IRAK-4 degrader, the method comprising a) incubating the innate immune cells from the individual with the gene therapy agent, b) analyzing the innate immune cells for the expression of one or more cytokines, wherein expression of a cytokine signature following incubation with the gene therapy agent identifies an individual for treatment with a gene therapy agent and an IRAK degrader, c) selecting the individual identified in step b) for treatment with a gene therapy agent and an IRAK degrader. In some embodiments, the methods further comprise the steps of d) administering an IRAK degrader to the individual identified in step b), and e) administering the gene therapy agent to the individual identified in step b). In some embodiments, the innate immune cells are dendritic cells, monocytes, macrophages, or natural killer (NK) cells. In some embodiments, the method further includes the steps of isolating innate immune cells from the individual prior to incubating the innate immune cells with the gene therapy agent. In some embodiments, the method further includes the steps of isolating monocytes from the individual and, incubating the monocytes in dendritic cell culture media to derive dendritic cells from the monocytes prior to incubating the dendritic cells with the gene therapy agent.

### General Techniques

The techniques and procedures described or referenced herein are generally well understood and commonly employed using conventional methodology by those skilled in the art, such as, for example, the widely utilized methodologies described in Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F. M. Ausubel, et al. eds., 2003); the series Methods in Enzymology (Academic Press, Inc.); PCR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6^{th} ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C. A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 2011).

### Definitions

As used herein, the term “degrader” is a heterobifunctional compound that binds to and/or inhibits both an IRAK kinase and an E3 ligase with measurable affinity resulting in the ubiqitination and subsequent degradation of the IRAK kinase. In certain embodiments, a degrader has an DC₅₀ of less than about 50 μM, less than about 1 μM, less than about 500 nM, less than about 100 nM, less than about 10 nM, or less than about 1 nM. As used herein, the term “monovalent” refers to a degrader compound without an appended E3 ligase binding moiety.

As used herein, the term “inhibitor” in reference to IRAK is a compound that binds to and/or inhibits an IRAK kinase with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM.

As used herein, the term “modulator” in reference to IRAK is a compound that stimulates, delays, inhibits and/or or suppresses (completely or partially) the activity of an IRAK kinase.

As used herein, the term “gene therapy agent” refers to a nucleic acid (e.g., expression construct, miRNA, antisense, shRNA, siRNA) or a nucleic acid in combination with an agent used to deliver the nucleic acid to an individual or a cell to modify or manipulate the expression of one or more nucleic acids (e.g., gene, mRNA) in an individual or a cell to alter the biological propertied of living cells. Examples of gene therapy agents include, but are not limited to, viral vectors (e.g., adeno-associated virus, adenovirus, lentivirus, Herpes simples virus, baculavirus), bacterial vectors, and non-viral vectors (e.g., lipid nanoparticles encapsulating a therapeutic nucleic acid or plasmid DNAs (e.g., close ended DNA) comprising a therapeutic nucleic acid and/or encoding a therapeutic polypeptide).

As used herein, the term “gene therapy agent” refers to an agent used to deliver nucleic acid to an individual or a cell to modify or manipulate the expression of one or more genes in an individual or a cell to alter the biological propertied of living cells. Examples of gene therapy agents include, but are not limited to, viral vectors (e.g., adeno-associated virus, adenovirus, lentivirus, Herpes simples virus, baculovirus), bacterial vectors, and non-viral vectors (e.g., lipid nanoparticles encapsulating a therapeutic nucleic acid or plasmid DNAs comprising a therapeutic nucleic acid and/or encoding a therapeutic polypeptide).

A “vector,” as used herein, refers to a recombinant plasmid or virus that comprises a nucleic acid to be delivered into a host cell, either in vitro or in vivo.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the nucleic acid can comprise sugars and phosphate groups (as may typically be found in RNA or DNA), or modified or substituted sugar or phosphate groups. Alternatively, the backbone of the nucleic acid can comprise a polymer of synthetic subunits such as phosphoramidates and thus can be an oligodeoxynucleoside phosphoramidate (P—NH₂) or a mixed phosphoramidate-phosphodiester oligomer. In addition, a double-stranded nucleic acid can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-translational modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

A “recombinant viral vector” refers to a recombinant polynucleotide vector comprising one or more heterologous sequences (i.e., nucleic acid sequence not of viral origin). In the case of recombinant AAV vectors, the recombinant nucleic acid is flanked by at least one, e.g., two, inverted terminal repeat sequences (ITRs).

A “recombinant AAV vector (rAAV vector)” refers to a polynucleotide vector comprising one or more heterologous sequences (i.e., nucleic acid sequence not of AAV origin) that are flanked by at least one, e.g., two, AAV inverted terminal repeat sequences (ITRs). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. A rAAV vector can be in any of a number of forms, including, but not limited to, plasmids, linear artificial chromosomes, complexed with lipids, encapsulated within liposomes, and, in embodiments, encapsidated in a viral particle, particularly an AAV particle. A rAAV vector can be packaged into an AAV virus capsid to generate a “recombinant adeno-associated viral particle (rAAV particle)”.

An “rAAV virus” or “rAAV viral particle” refers to a viral particle composed of at least one AAV capsid protein and an encapsidated rAAV vector genome.

A “recombinant adenoviral vector” refers to a polynucleotide vector comprising one or more heterologous sequences (i.e., nucleic acid sequence not of adenovirus origin) that are flanked by at least one adenovirus inverted terminal repeat sequence (ITR). In some embodiments, the recombinant nucleic acid is flanked by two inverted terminal repeat sequences (ITRs). Such recombinant viral vectors can be replicated and packaged into infectious viral particles when present in a host cell that is expressing essential adenovirus genes deleted from the recombinant viral genome (e.g., E1 genes, E2 genes, E4 genes, etc.). When a recombinant viral vector is incorporated into a larger polynucleotide (e.g., in a chromosome or in another vector such as a plasmid used for cloning or transfection), then the recombinant viral vector may be referred to as a “pro-vector” which can be “rescued” by replication and encapsidation in the presence of adenovirus packaging functions. A recombinant viral vector can be in any of a number of forms, including, but not limited to, plasmids, linear artificial chromosomes, complexed with lipids, encapsulated within liposomes, and encapsidated in a viral particle, for example, an adenovirus particle. A recombinant viral vector can be packaged into an adenovirus virus capsid to generate a “recombinant adenoviral particle.”

A “recombinant lentivirus vector” refers to a polynucleotide vector comprising one or more heterologous sequences (i.e., nucleic acid sequence not of lentivirus origin) that are flanked by at least one lentivirus terminal repeat sequences (LTRs). In some embodiments, the recombinant nucleic acid is flanked by two lentiviral terminal repeat sequences (LTRs). Such recombinant viral vectors can be replicated and packaged into infectious viral particles when present in a host cell that has been infected with a suitable helper functions. A recombinant lentiviral vector can be packaged into a lentivirus capsid to generate a “recombinant lentiviral particle.”

A “recombinant herpes simplex vector (recombinant HSV vector)” refers to a polynucleotide vector comprising one or more heterologous sequences (i.e., nucleic acid sequence not of HSV origin) that are flanked by HSV terminal repeat sequences. Such recombinant viral vectors can be replicated and packaged into infectious viral particles when present in a host cell that has been infected with a suitable helper functions. When a recombinant viral vector is incorporated into a larger polynucleotide (e.g., in a chromosome or in another vector such as a plasmid used for cloning or transfection), then the recombinant viral vector may be referred to as a “pro-vector” which can be “rescued” by replication and encapsidation in the presence of HSV packaging functions. A recombinant viral vector can be in any of a number of forms, including, but not limited to, plasmids, linear artificial chromosomes, complexed with lipids, encapsulated within liposomes, and encapsidated in a viral particle, for example, an HSV particle. A recombinant viral vector can be packaged into an HSV capsid to generate a “recombinant herpes simplex viral particle.”

“Solid lipid nanoparticles” (SLNs, sLNPs), or “lipid nanoparticles” (LNPs) as used herein refer to nanoparticles composed of lipids. In some examples, there is only one phospholipid layer and the bulk of the interior of the particle is composed of lipophilic substance. Payloads such as nucleic acids can be embedded in the interior. In some examples, the lipid nanoparticle is a liposome, which comprise a lipid bilayer.

As used herein, the term “improving” as it relates to gene therapy may refer to the act of boosting, heightening, lengthening or otherwise increasing the expression of the therapeutic gene payload of a gene therapy agent. In some embodiments, an improved gene therapy is one where expression of the therapeutic gene payload of the gene therapy agent administered with an IRAK modulator is increased by greater than any of about 10%, 25%, 50%, 75%, or 100% compared to gene therapy administered without the IRAK modulator. In some embodiments, an improved gene therapy is one where time of expression of the therapeutic gene payload of the gene therapy agent administered with an IRAK degrader is lengthened by greater than any of about 10%, 25%, 50%, 75%, or 100% compared to gene therapy administered without the IRAK degrader. In some examples, a gene therapy is improved by decreasing an immune response (e.g., an innate immune response) to the gene therapy agent. In some embodiments, an improved gene therapy is one where an immune response to gene therapy agent administered with an IRAK degrader is decreased by greater than any of about 10%, 25%, 50%, 75%, or 100% compared to gene therapy administered without the IRAK degrader. In some embodiments, the decrease in an immune response to a gene therapy agent is measured as a decrease in a cytokine signature following exposure of the gene therapy agent to immune cells in the presence of an IRAK degrader compared to exposure of the gene therapy agent to immune cells in the absence of an IRAK degrader.

As used herein, the term “modulating” as it refers to gene therapy may refer to the act of changing, altering, varying, improving or otherwise modifying the presence, or an activity of, a gene therapy agent. For example, modulating an immune response to a gene therapy agent may refer to any act leading to changing, altering, varying, improving or otherwise modifying an immune response to the gene therapy agent (e.g., decreasing, delaying and/or eliminating an immune response (e.g., an innate immune response) to the gene therapy agent).

As used herein, the term “cytokine signature” as it relates to an immune response (e.g., innate immune response) to a gene therapy agent refers to the increased expression of one or more cytokines following exposure of an innate immune cell to a gene therapy agent. In some examples, the cytokines of the cytokine signature are specific to a TLR pathway (e.g., a TLR2, TLR3, TLR4 or TLR9 pathway).

Innate immune cells are white blood cells that mediate innate immunity and include basophils, dendritic cells, eosinophils, Langerhans cells, mast cells, monocytes and macrophages, neutrophils and NK cells. Different AAV capsids can enter these innate immune cells with different efficiencies often referred to as transduction efficiency. Some serotypes such as AAV1 are efficient at transducing certain immune cells like monocytes whereas other AAVs like AAV6 are efficient at transducing cells like dendritic cells (Grimm, D et al., J. Virol., 2008, 82(12):5887-5911). AAVs upon cell entry can evoke an immune response. The magnitude of this immune response is dependent on AAV serotype and cell type. Once AAVs transduce a host immune cell they can engage immune receptors such as TLRs (e.g., TLR9). Several studies using mouse models reveal that TLR9 is a key DNA sensor contributing to AAV immunogenicity (Zhu, J et al., J Clin Invest. 2009; 119(8):2388-2398; Ashley S N et al., Cell. Immunol. 2019, 346:103997). Once these TLRs are activated by viruses they secrete cytokines that establish an anti-viral state within the infected cell and alert the neighboring cells. (Carty, M and Bowie, AG, Clin Exp Immunol, 2010, 161(3):397-406; Lester, SN and Li, K, J Mol Biol. 2014; 426(6):1246-1264; Fitzgerald, K A and Kagan, J C, Cell, 2020 180(6):1044-1066).

These cytokines are also responsible for activating the adaptive immune system that comprises B cells and T cells which produce antibodies and generate cytotoxicity to kill the viral infected cells respectively. As used herein, the upregulation or downregulation of certain subset of cytokines is referred to as a “cytokine signature”. These cytokine signatures comprising three or more cytokines can be used as predictive markers for diseases and success of therapies. Examples of cytokine signatures are found in Zuniga, J et al., Int. J. Infect. Diseases, 2020, 94:4-11, Bergamaschi, C et al., Cell Reports, 2021, 36:109504; Del Valle, D M et al., Nat. Med. 2020, 26:1636-1643.

“Heterologous” means derived from a genotypically distinct entity from that of the rest of the entity to which it is compared or into which it is introduced or incorporated. For example, a nucleic acid introduced by genetic engineering techniques into a different cell type is a heterologous nucleic acid (and, when expressed, can encode a heterologous polypeptide). Similarly, a cellular sequence (e.g., a gene or portion thereof) that is incorporated into a viral vector is a heterologous nucleotide sequence with respect to the vector.

The term “transgene” refers to a nucleic acid that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome. In another aspect, it may be transcribed into a molecule that mediates RNA interference, such as siRNA.

The terms “genome particles (gp),” “genome equivalents,” or “genome copies” as used in reference to a viral titer, refer to the number of virions containing the recombinant AAV DNA genome, regardless of infectivity or functionality. The number of genome particles in a particular vector preparation can be measured by procedures such as described in the Examples herein, or for example, in Clark et al. (1999) Hum. Gene Ther., 10:1031-1039; Veldwijk et al. (2002) Mol. Ther., 6:272-278.

The terms “infection unit (iu),” “infectious particle,” or “replication unit,” as used in reference to a viral titer, refer to the number of infectious and replication-competent recombinant AAV vector particles as measured by the infectious center assay, also known as replication center assay, as described, for example, in McLaughlin et al. (1988) J. Virol., 62:1963-1973.

The term “transducing unit (tu)” as used in reference to a viral titer, refers to the number of infectious recombinant AAV vector particles that result in the production of a functional transgene product as measured in functional assays such as described in Examples herein, or for example, in Xiao et al. (1997) Exp. Neurobiol., 144:113-124; or in Fisher et al. (1996) J. Virol., 70:520-532 (LFU assay).

An “inverted terminal repeat” or “ITR” sequence is a term well understood in the art and refers to relatively short sequences found at the termini of viral genomes which are in opposite orientation.

An “AAV inverted terminal repeat (ITR)” sequence, a term well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C′ and D regions), allowing intrastrand base-pairing to occur within this portion of the ITR.

A “terminal resolution sequence” or “trs” is a sequence in the D region of the AAV ITR that is cleaved by AAV rep proteins during viral DNA replication. A mutant terminal resolution sequence is refractory to cleavage by AAV rep proteins. “AAV helper functions” refer to functions that allow AAV to be replicated and packaged by a host cell. AAV helper functions can be provided in any of a number of forms, including, but not limited to, helper virus or helper virus genes which aid in AAV replication and packaging. Other AAV helper functions are known in the art such as genotoxic agents.

“AAV helper functions” refer to functions that allow AAV to be replicated and packaged by a host cell. AAV helper functions can be provided in any of a number of forms, including, but not limited to, helper virus or helper virus genes which aid in AAV replication and packaging. Other AAV helper functions are known in the art such as genotoxic agents.

A “helper virus” for AAV refers to a virus that allows AAV (which is a defective parvovirus) to be replicated and packaged by a host cell. A number of such helper viruses have been identified, including adenoviruses, herpesviruses, poxviruses such as vaccinia, and baculovirus. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Baculoviruses available from depositories include Autographa californica nuclear polyhedrosis virus.

“Percent (%) sequence identity” with respect to a reference polypeptide or nucleic acid sequence is defined as the percentage of amino acid residues or nucleotides in a candidate sequence that are identical with the amino acid residues or nucleotides in the reference polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various ways that are within the skill in the art, for instance, using publicly available computer software programs, for example, those described in Current Protocols in Molecular Biology (Ausubel et al., eds., 1987), Supp. 30, section 7.7.18, Table 7.7.1, and including BLAST, BLAST-2, ALIGN or Megalign (DNASTAR) software. A potential alignment program is ALIGN Plus (Scientific and Educational Software, Pennsylvania). Those skilled in the art can determine appropriate parameters for measuring alignment, including any algorithms needed to achieve maximal alignment over the full length of the sequences being compared. For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z, where W is the number of nucleotides scored as identical matches by the sequence alignment program in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

An “effective amount” of an agent refers to an amount effective, at dosages and for periods of time necessary, to achieve the desired therapeutic result. For example, an effective amount of a gene therapy agent refers to an amount effective, at dosages and for periods of time necessary, to achieve the desired gene therapeutic result. In another example, an effective amount of an IRAK degrader may refer to an amount effective, at dosages and for periods of time necessary, to achieve the desired result of improved gene therapy.

A “therapeutically effective amount” of a substance/molecule of the invention, (e.g., a gene therapy agent and/or an IRAK degrader) may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule are outweighed by the therapeutically beneficial effects.

The term “suitable control” as it refers to a cytokine signature is the expression of the cytokines in the cytokine signature from innate immune cells that are not incubated with the gene therapy agent or the expression of the cytokines in the cytokine signature from innate immune cells prior to incubation with the gene therapy agent.

Administration “in combination with” as it related to a gene therapy agent and an IRAK degrader includes simultaneous (concurrent) and consecutive or sequential administration in any order of the gene therapy agent and the IRAK degrader . . . .

The term “concurrently” is used herein to refer to administration of a gene therapy agent and an IRAK degrader, where at least part of the administration overlaps in time. Accordingly, concurrent administration includes a dosing regimen when the administration of a gene therapy agent or an IRAK degrader continues after discontinuing the administration of the other agent/degrader.

As used herein, “in conjunction with” refers to administration of one treatment modality (in addition to another treatment modality. As such, “in conjunction with” refers to administration of one treatment modality (a gene therapy agent or an IRAK degrader) before, during or after administration of the other treatment modality to the individual.

An “isolated” molecule (e.g., nucleic acid or protein) or cell means it has been identified and separated and/or recovered from a component of its natural environment.

Reference to “about” a value or parameter herein includes (and describes) embodiments that are directed to that value or parameter per se. For example, description referring to “about X” includes description of “X.”

As used herein, the singular form of the articles “a,” “an,” and “the” includes plural references unless indicated otherwise.

It is understood that aspects and embodiments of the invention described herein include “comprising,” “consisting,” and/or “consisting essentially of” aspects and embodiments.

### Methods of Treatment

In some aspects, the invention provides methods for delivering nucleic acid to a cell of an individual, the method comprising a) administering an IRAK degrader to the individual, and b) administering a gene therapy agent to the individual. In some aspects, the invention provides methods for treating an individual in need thereof, the method comprising a) administering an IRAK degrader to the individual, and b) administering a gene therapy agent to the individual. In some aspects, the invention provides methods for improving gene therapy in an individual, the method comprising a) administering an IRAK degrader to the individual, and b) administering a gene therapy agent to the individual. In some aspects, the invention provides methods for modulating an immune response to a gene therapy agent, the method comprising a) administering an IRAK degrader to the individual, and b) administering a gene therapy agent to the individual. In some aspects, the invention provides methods for suppressing an immune response to a gene therapy agent, the method comprising a) administering an IRAK degrader to the individual, and b) administering a gene therapy agent to the individual. In some aspects, the invention provides methods for inducing tolerance to a gene therapy agent, the method comprising a) administering an IRAK degrader to the individual, and b) administering a gene therapy agent to the individual. In some embodiments, the IRAK degrader modulates the activity of an IRAK protein kinase. In some embodiments, the IRAK degrader modulates the activity of an IRAK-4 protein kinase. In some embodiments, the gene therapy agent is a viral gene therapy agent (e.g., a viral vector) or a non-viral gene therapy agent (e.g., a lipid nanoparticle comprising a non-viral gene therapy agent). In some embodiments, the gene therapy agent is an adeno-associated virus (AAV) vector, an adenovirus vector, a lentivirus vector, or a herpes simplex virus (HSV) vector.

In some embodiments of the invention, the gene therapy agent (e.g., an AAV particle, an adenovirus particle, a lentivirus particle, a HSV particle, or a lipid nanoparticle) may be administered to a particular tissue of interest, or it may be administered systemically. In some embodiments, an effective amount of the gene therapy agent may be administered to the subject. In some embodiments, an effective amount of the gene therapy agent may be administered parenterally. Parenteral routes of administration may include without limitation intravenous, intraperitoneal, intraosseous, intra-arterial, intracerebral, intramuscular, intrathecal, subcutaneous, intracerebroventricular, intrahepatic, and so forth. In some embodiments, an effective amount of the gene therapy agent may be administered through one route of administration. In some embodiments, an effective amount of the gene therapy agent may be administered through a combination of or multiple routes of administration (e.g., two, three etc.). In some embodiments, an effective amount of the gene therapy agent is administered to one location. In other embodiments, an effective amount of the gene therapy agent may be administered to more than one location.

An effective amount of gene therapy agent (e.g., an AAV particle, an adenovirus particle, a lentivirus particle, a HSV particle, or a lipid nanoparticle) is administered, depending on the objectives of treatment. For example, where a low percentage of transduction or transfection can achieve the desired therapeutic effect, then the objective of treatment is generally to meet or exceed this level of transduction or transfection. In some instances, this level of transduction or transfection can be achieved by transduction or transfection of only about 1 to 5% of the target cells of the desired tissue type, in some embodiments at least about 20% of the cells of the desired tissue type, in some embodiments at least about 50%, in some embodiments at least about 80%, in some embodiments at least about 95%, in some embodiments at least about 99% of the cells of the desired tissue type. The gene therapy agent may be administered by one or more administrations, either during the same procedure or spaced apart by days, weeks, months, or years. One or more of any of the routes of administration described herein may be used. In some embodiments, multiple gene therapy agents may be used to treat the human; for example, an AAV vector and a lentiviral vector.

Methods to identify cells transduced or transfected by gene therapy agents are known in the art; for example, immunohistochemistry or the use of a marker such as enhanced green fluorescent protein can be used to detect transduction or transfection cells by the gene therapy agent.

In some embodiments, an effective amount of gene therapy agent (e.g., an AAV particle, an adenovirus particle, a lentivirus particle, a HSV particle, or a lipid nanoparticle) is administered to more than one location simultaneously or sequentially. In other embodiments, an effective amount of the gene therapy agent is administered to a single location more than once (e.g., repeated). In some embodiments, multiple injections of the gene therapy agent are no more than one hour, two hours, three hours, four hours, five hours, six hours, nine hours, twelve hours or 24 hours apart.

In some embodiments, the methods comprise administering an effective amount of a pharmaceutical composition comprising a gene therapy agent to treat an individual in need of gene therapy treatment. In some embodiments, the viral titer of the viral particles (e.g., rAAV particles) is at least about any of 5×10¹², 6×10¹², 7×10¹², 8×10¹², 9×10¹², 10×10¹², 11×10¹², 15×10¹², 20×10¹², 25×10¹², 30×10¹², or 50×10¹² genome copies/mL. In some embodiments, the viral titer of the viral particles (e.g., rAAV particles) is about any of 5×10¹² to 6×10¹², 6×10¹² to 7×10¹², 7×10¹² to 8×10¹², 8×10¹² to 9×10¹², 9×10¹² to 10×10¹², 10×10¹² to 11×10¹², 11×10¹² to 15×10¹², 15×10¹² to 20×10¹², 20×10¹² to 25×10¹², 25×10¹² to 30×10¹², 30×10¹² to 50×10¹², or 50×10¹² to 100×10¹² genome copies/mL. In some embodiments, the viral titer of the viral particles (e.g., rAAV particles) is about any of 5×10¹² to 10×10¹², 10×10¹² to 25×10¹², or 25×10¹² to 50×10¹²genome copies/mL. In some embodiments, the viral titer of the viral particles (e.g., rAAV particles) is at least about any of 5×10⁹, 6×10⁹, 7×10⁹, 8×10⁹, 9×10⁹, 10×10⁹, 11×10⁹, 15×10⁹, 20×10⁹, 25×10⁹, 30×10⁹, or 50×10⁹ transducing units/mL. In some embodiments, the viral titer of the viral particles (e.g., rAAV particles) is about any of 5×10⁹ to 6×10⁹, 6×10⁹ to 7×10⁹, 7×10⁹ to 8×10⁹, 8 ×10⁹ to 9×10⁹, 9×10⁹ to 10×10⁹, 10×10⁹ to 11×10⁹, 11×10⁹ to 15×10⁹, 15×10⁹ to 20×10⁹, 20 ×10⁹ to 25×10⁹, 25×10⁹ to 30×10⁹, 30×10⁹ to 50×10⁹ or 50×10⁹ to 100×10⁹ transducing units/mL. In some embodiments, the viral titer of the viral particles (e.g., rAAV particles) is about any of 5×10⁹ to 10×10⁹, 10×10⁹ to 15×10⁹, 15×10⁹ to 25×10⁹, or 25×10⁹ to 50×10⁹ transducing units/mL. In some embodiments, the viral titer of the viral particles (e.g., rAAV particles) is at least any of about 5×10¹⁰, 6×10¹⁰, 7×10¹⁰, 8×10¹⁰, 9×10¹⁰, 10×10¹⁰, 11×10¹⁰, 15×10¹⁰, 20×10¹⁰, 25×10¹⁰, 30×10¹⁰, 40×10¹⁰, or 50×10¹⁰ infectious units/mL. In some embodiments, the viral titer of the viral particles (e.g., rAAV particles) is at least any of about 5×10¹⁰ to 6×10¹⁰, 6×10¹⁰ to 7×10¹⁰, 7×10¹⁰ to 8×10¹⁰, 8×10¹⁰ to 9×10¹⁰, 9×10¹⁰ to 10×10¹⁰, 10×10¹⁰ to 11×10¹⁰, 11×10¹⁰ to 15×10¹⁰, 15×10¹⁰ to 20×10¹⁰, 20×10¹⁰ to 25×10¹⁰, 25×10¹⁰ to 30×10¹⁰, 30×10¹⁰ to 40×10¹⁰, 40×10¹⁰ to 50×10¹⁰, or 50×10¹⁰ to 100×10¹⁰ infectious units/mL. In some embodiments, the viral titer of the viral particles (e.g., rAAV particles) is at least any of about 5×10¹⁰ to 10×10¹⁰, 10×10¹⁰ to 15×10¹⁰, 15×10¹⁰ to 25×10¹⁰, or 25×10¹⁰ to 50×10¹⁰ infectious units/mL.

In some embodiments, the dose of gene therapy agent (e.g., an AAV particle, an adenovirus particle, a lentivirus particle, a HSV particle, or a lipid nanoparticle) administered to the individual is at least about any of 1×10⁸ to about 6×10¹³ genome copies/kg of body weight. In some embodiments, the dose of gene therapy agent administered to the individual is about any of 1×10⁸ to about 6×10¹³ genome copies/kg of body weight.

In some embodiments, the total amount of the gene therapy agent (e.g., an AAV particle, an adenovirus particle, a lentivirus particle, a HSV particle, or a lipid nanoparticle) administered to the individual is at least about any of 1×10⁹ to about 1×10¹⁴ genome copies. In some embodiments, the total amount of the gene therapy agent administered to the individual is about any of 1×10⁹ to about 1×10¹⁴ genome copies.

Compositions of the invention comprising the gene therapy (e.g., an AAV particle, an adenovirus particle, a lentivirus particle, a HSV particle, or a lipid nanoparticle) can be used either alone or in combination with one or more additional therapeutic agents in addition to the IRAK degrader. The interval between sequential administration can be in terms of at least (or, alternatively, less than) minutes, hours, or days.

In some embodiments of the invention, compositions comprising the IRAK degrader (e.g., an IRAK-4 degrader) may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a nontoxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

The amount of IRAK degrader (e.g., an IRAK-4 degrader may be combined with the carrier materials to produce a composition (e.g., a pharmaceutical composition) in a single dosage form will vary depending upon the individual and the particular mode of administration. In some embodiments, compositions comprising the IRAK degrader are formulated so that a dosage of between about 0.01 mg/kg to about 100 mg/kg body weight of the IRAK degrader is administered to the individual. In some embodiments, the IRAK degrader is administered orally or parenterally at dosage levels of any of about of 0.01 mg/kg to about 100 mg/kg, about of 0.01 mg/kg to about 75 mg/kg, about of 0.01 mg/kg to about 500 mg/kg, about of 0.01 mg/kg to about 25 mg/kg, about of 0.01 mg/kg to about 10 mg/kg, about of 0.01 mg/kg to about 5 mg/kg, about of 0.01 mg/kg to about 1.0 mg/kg, about of 1.0 mg/kg to about 100 mg/kg, about of 1.0 mg/kg to about 75 mg/kg, about of 1.0 mg/kg to about 50 mg/kg, about of 1.0 mg/kg to about 25 mg/kg, about of 1.0 mg/kg to about 10 mg/kg, about of 1.0 mg/kg to about 5 mg/kg, about of 10 mg/kg to about 100 mg/kg, about of 10 mg/kg to about 75 mg/kg, about of 10 mg/kg to about 50 mg/kg, about of 10 mg/kg to about 25 mg/kg, about of 25 mg/kg to about 100 mg/kg, about of 25 mg/kg to about 75 mg/kg, about of 25 mg/kg to about 50 mg/kg, about of 50 mg/kg to about 100 mg/kg, about of 50 mg/kg to about 75 mg/kg, or about of 75 mg/kg to about 100 mg/kg body weight of the individual. In some embodiments, the IRAK degrader is administered orally or parenterally at dosage levels of more than about any of 0.01 mg/kg, 1.0 mg/kg, 5 mg/kg, 25 mg/kg, 50 mg/kg, 75 mg/kg, 100 mg/kg body, 200 mg/kg body, 300 mg/kg body, 400 mg/kg body, or 500 mg/kg body weight of the individual.

Liquid dosage forms for oral administration of the IRAK degrader (e.g., an IRAK-4 degrader include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1, 3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions or the IRAK-4 degrader may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations or the IRAK-4 degrader can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration of the IRAK-4 degrader include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The IRAK-4 degrader can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

In some embodiments, the IRAK degrader (e.g., an IRAK-4 degrader is administered before administration of the gene therapy agent (e.g., an AAV particle, an adenovirus particle, a lentivirus particle, a HSV particle, or a lipid nanoparticle). In some embodiments, the IRAK degrader is administered to the individual about any of 1 hour, 2 hours, 3 hours, 4 hours, 5 hours, 6 hours, 12 hours, 18 hours, 24 hours, 36 hours, 48 hours, 3 days, 4 days, 5 days, 6 days, one week or more than one week before administration of the gene therapy agent. In some embodiments, the IRAK degrader is administered to the individual less than about any of 1 hour, 2 hours, 3 hours, 4 hours, 5 hours, 6 hours, 12 hours, 18 hours, 24 hours, 36 hours, 48 hours, 3 days, 4 days, 5 days, 6 days, or one week before administration of the gene therapy agent. In some embodiments, the IRAK degrader and the gene therapy agent are administered at about the same time (e.g., within about one hour). In some embodiments, the IRAK degrader is administered after administration of the gene therapy agent. In some embodiments, the IRAK degrader is administered to the individual about any of 1 hour, 2 hours, 3 hours, 4 hours, 5 hours, 6 hours, 12 hours, 18 hours, 24 hours, 36 hours, 48 hours, 3 days, 4 days, 5 days, 6 days, one week or more than one week after administration of the gene therapy agent. In some embodiments, the IRAK degrader is administered to the individual less than about any of 1 hour, 2 hours, 3 hours, 4 hours, 5 hours, 6 hours, 12 hours, 18 hours, 24 hours, 36 hours, 48 hours, 3 days, 4 days, 5 days, 6 days, or one week after administration of the gene therapy agent.

In some embodiments, the gene therapy agent (e.g., an AAV particle, an adenovirus particle, a lentivirus particle, a HSV particle, or a lipid nanoparticle) is used in conjunction with the IRAK degrader (e.g., an IRAK-4 degrader to treat a disease or disorder suitable for treatment by gene therapy. In some embodiments, the disease or disorder is a monogenic disease or disorder.

In some embodiments, the gene therapy agent (e.g., an AAV particle, an adenovirus particle, a lentivirus particle, a HSV particle, or a lipid nanoparticle) is used in conjunction with the IRAK degrader (e.g., an IRAK-4 degrader) to treat a disorder of the CNS. Non-limiting disorders of the CNS include stroke, Huntington's disease, epilepsy, Parkinson's disease, Lou Gehrig's disease (also known as amyotrophic lateral sclerosis), Alzheimer's disease, corticobasal degeneration or CBD, corticogasal ganglionic degeneration or CBGD, frontotemporal dementia or FTD, progressive supranuclear palsy or PSP, multiple system atrophy or MSA, cancer of the brain, and lysosomal storage diseases (LSD). Other non-limiting examples of disorders of the invention that may be treated by a gene therapy in conjunction with an IRAK degrader include traumatic brain injury, enzymatic dysfunction disorders, psychiatric disorders (including post-traumatic stress syndrome), neurodegenerative diseases, and cognitive disorders (including dementias, autism, and depression), and enzymatic dysfunction disorders include without limitation leukodystrophies (including Canavan's disease).

In some embodiments, the gene therapy agent (e.g., an AAV particle, an adenovirus particle, a lentivirus particle, a HSV particle, or a lipid nanoparticle) is used in conjunction with the IRAK degrader (e.g., an IRAK-4 degrader) to treat a lysosomal storage disease. As is commonly known in the art, lysosomal storage disease are rare, inherited metabolic disorders characterized by defects in lysosomal function. Such disorders are often caused by a deficiency in an enzyme required for proper mucopolysaccharide, glycoprotein, and/or lipid metabolism, leading to a pathological accumulation of lysosomally stored cellular materials. Non-limiting examples of lysosomal storage diseases of the invention that may be treated by a therapeutic polypeptide or therapeutic nucleic acid of the invention include Gaucher disease type 2 or type 3, GM1 gangliosidosis, Hunter disease, Krabbe disease, a mannosidosis disease, β mannosidosis disease, metachromatic leukodystrophy disease, mucolipidosisII/III disease, Niemann-Pick A disease, Niemann-Pick C disease, Pompe disease, Sandhoff disease, Sanfilippo A disease, Sanfilippo B disease, Sanfilippo C disease, Sanfilippo D disease, Schindler disease, Sly disease, Tay-Sachs disease, and Wolman disease.

In some embodiments, the gene therapy agent (e.g., an AAV particle, an adenovirus particle, a lentivirus particle, a HSV particle, or a lipid nanoparticle) is used in conjunction with the IRAK degrader (e.g., an IRAK-4 degrader) to treat hemophilia A, hemophilia B, age related macular degeneration, diabetic retinopathy, glaucoma, muscular dystrophy, X-Linked Myotubular Myopathy, spinal muscular atrophy, Leber's congenital amaurosis, choroideremia, Leber hereditary optic neuropathy, ornithine transcarbamylase (OTC) deficiency, citrullinemia type 1, phenylketonuria (PKU), adrenoleukodystrophy, sickle cell disease, muscular dystrophy, or beta thalassemia.

In some aspects, the invention provides a composition for use in the manufacture of a medicament for delivering nucleic acid to a cell of an individual in need thereof, wherein the composition comprises a gene therapy agent (e.g., an AAV particle, an adenovirus particle, a lentivirus particle, a HSV particle, or a lipid nanoparticle), and wherein the composition is formulated for use in combination with an IRAK degrader (e.g., an IRAK-4 degrader). In some aspects, the invention provides a composition for use in the manufacture of a medicament for delivering nucleic acid to a cell of an individual in need thereof, wherein the composition comprises an IRAK degrader (e.g., an IRAK-4 degrader), and wherein the composition is formulated for use in combination with a gene therapy agent (e.g., an AAV particle, an adenovirus particle, a lentivirus particle, a HSV particle, or a lipid nanoparticle).

In some aspects, the invention provides a composition for use in the manufacture of a medicament for treating an individual in need of gene therapy, wherein the composition comprises a gene therapy agent (e.g., an AAV particle, an adenovirus particle, a lentivirus particle, a HSV particle, or a lipid nanoparticle), and wherein the composition is formulated for use in combination with an IRAK degrader (e.g., an IRAK-4 degrader). In some aspects, the invention provides a composition for use in the manufacture of a medicament for treating an individual in need of gene therapy, wherein the composition comprises an IRAK degrader (e.g., an IRAK-4 degrader), and wherein the composition is formulated for use in combination with a gene therapy agent (e.g., an AAV particle, an adenovirus particle, a lentivirus particle, a HSV particle, or a lipid nanoparticle).

In some aspects, the invention provides a composition for use in the manufacture of a medicament for modulating an immune response to gene therapy in an individual in need of gene therapy, wherein the composition comprises a gene therapy agent (e.g., an AAV particle, an adenovirus particle, a lentivirus particle, a HSV particle, or a lipid nanoparticle), and wherein the composition is formulated for use in combination with an IRAK degrader (e.g., an IRAK-4 degrader). In some aspects, the invention provides a composition for use in the manufacture of a medicament for modulating an immune response to gene therapy in an individual, wherein the composition comprises an IRAK degrader (e.g., an IRAK-4 degrader), and wherein the composition is formulated for use in combination with a gene therapy agent (e.g., an AAV particle, an adenovirus particle, a lentivirus particle, a HSV particle, or a lipid nanoparticle).

In some aspects, the invention provides a composition for use in the manufacture of a medicament for suppressing an immune response to gene therapy in an individual in need of gene therapy, wherein the composition comprises a gene therapy agent (e.g., an AAV particle, an adenovirus particle, a lentivirus particle, a HSV particle, or a lipid nanoparticle), and wherein the composition is formulated for use in combination with an IRAK degrader (e.g., an IRAK-4 degrader). In some aspects, the invention provides a composition for use in the manufacture of a medicament for suppressing an immune response to gene therapy in an individual, wherein the composition comprises an IRAK degrader (e.g., an IRAK-4 degrader), and wherein the composition is formulated for use in combination with a gene therapy agent (e.g., an AAV particle, an adenovirus particle, a lentivirus particle, a HSV particle, or a lipid nanoparticle).

In some aspects, the invention provides a composition for use in the manufacture of a medicament for improving gene therapy in an individual in need of gene therapy, wherein the composition comprises a gene therapy agent (e.g., an AAV particle, an adenovirus particle, a lentivirus particle, a HSV particle, or a lipid nanoparticle), and wherein the composition is formulated for use in combination with an IRAK degrader (e.g., an IRAK-4 degrader). In some aspects, the invention provides a composition for use in the manufacture of a medicament for improving gene therapy in an individual, wherein the composition comprises an IRAK degrader (e.g., an IRAK-4 degrader), and wherein the composition is formulated for use in combination with a gene therapy agent (e.g., an AAV particle, an adenovirus particle, a lentivirus particle, a HSV particle, or a lipid nanoparticle).

In some aspects, the invention provides a composition for use in the manufacture of a medicament for inducing tolerance to gene therapy in an individual in need of gene therapy, wherein the composition comprises a gene therapy agent (e.g., an AAV particle, an adenovirus particle, a lentivirus particle, a HSV particle, or a lipid nanoparticle), and wherein the composition is formulated for use in combination with an IRAK degrader (e.g., an IRAK-4 degrader). In some aspects, the invention provides a composition for use in the manufacture of a medicament for inducing tolerance to gene therapy in an individual, wherein the composition comprises an IRAK degrader (e.g., an IRAK-4 degrader), and wherein the composition is formulated for use in combination with a gene therapy agent (e.g., an AAV particle, an adenovirus particle, a lentivirus particle, a HSV particle, or a lipid nanoparticle).

In some aspects, the invention provides a gene therapy agent (e.g., an AAV particle, an adenovirus particle, a lentivirus particle, a HSV particle, or a lipid nanoparticle) for use in delivering nucleic acid to a cell of an individual in need thereof, wherein the gene therapy agent is used in combination with an IRAK degrader (e.g., an IRAK-4 degrader). In some aspects, the invention provides an IRAK degrader (e.g., an IRAK-4 degrader) for use in delivering nucleic acid to a cell of an individual in need thereof, wherein the IRAK degrader is used in combination with a gene therapy agent (e.g., an AAV particle, an adenovirus particle, a lentivirus particle, a HSV particle, or a lipid nanoparticle).

In some aspects, the invention provides a gene therapy agent (e.g., an AAV particle, an adenovirus particle, a lentivirus particle, a HSV particle, or a lipid nanoparticle) for use in treating an individual in need of gene therapy, wherein the gene therapy agent is used in combination with an IRAK degrader (e.g., an IRAK-4 degrader). In some aspects, the invention provides an IRAK degrader (e.g., an IRAK-4 degrader) for use in treating an individual in need of gene therapy, wherein the IRAK degrader is used in combination with a gene therapy agent (e.g., an AAV particle, an adenovirus particle, a lentivirus particle, a HSV particle, or a lipid nanoparticle).

In some aspects, the invention provides an IRAK degrader (e.g., an IRAK-4 degrader) for modulating an immune response to gene therapy in an individual in need of gene therapy, wherein the IRAK degrader is used in combination with a gene therapy agent (e.g., an AAV particle, an adenovirus particle, a lentivirus particle, a HSV particle, or a lipid nanoparticle).

In some aspects, the invention provides an IRAK degrader (e.g., an IRAK-4 degrader) for suppressing an immune response to gene therapy in an individual in need of gene therapy, wherein the IRAK degrader is used in combination with a gene therapy agent (e.g., an AAV particle, an adenovirus particle, a lentivirus particle, a HSV particle, or a lipid nanoparticle).

### IRAK Degraders

In some aspects, the invention provides methods for using IRAK degraders with gene therapy agents for improved gene therapy by inhibiting an immune response (e.g., innate immune response, adaptive immune response) to the gene therapy agent. IRAKs play a central role in the protective response to pathogens introduced into the human body by inducing acute inflammation followed by additional adaptive immune responses. IRAKs are essential components of the Interleukin-1 receptor signaling pathway and some Toll-like receptor signaling pathways. Toll-like receptors (TLRs) detect microorganisms by recognizing specific pathogen-associated molecular patterns (PAMPs) and IL-1R family members respond the interleukin-1 (IL-1) family cytokines. These receptors initiate an intracellular signaling cascade through adaptor proteins, primarily, MyD88.

The IRAK family is composed of IRAK-1, IRAK-2, and IRAK-4, which are expressed in a variety of human immune cell types and IRAK-M (also known as IRAK-3) whose expression is largely limited to monocytes and macrophages. All four IRAK family proteins contain an N-terminal death domain (DD), a ProST domain, and a centrally located kinase domain. IRAK-1, IRAK-2, and IRAK-M also contain a C-terminal domain. The DD serves as a platform that allows protein-protein interaction with other DD-containing proteins, the most important of which is the adaptor protein myeloid differentiation factor 88 (MyD88). The Inventors have hypothesized that blocking IRAK function should results in specific blockade of the TLR9 pathway that results in specific immune modulation. In some embodiments, an IRAK degrader is used to block the TLR9 pathway. In some embodiments, the IRAK degrader blocks TLR9 function.

In some embodiments, the IRAK degrader is a bifunctional compound that functions to recruit IRAK kinases to E3 Ubiquitin Ligase for degradation. In some embodiments, the IRAK degrader is a modulator of targeted ubiquitination of IRAK kinases.

Protein degraders are bifunctional compounds comprising three components: an E3 ubiquitin ligase ligand, a linker, and a ligand for a target protein of interest. They induce the formation of a ternary complex by simultaneously binding to both an E3 ligase and the target protein. Ternary complex formation effectively recruits the E3 ligase to polyubiquitinate the target of interest, inducing subsequent degradation by the proteasome. Degraders are attractive tools for use to induce selective protein knockdown in a reversible and tunable manner. In some embodiments, the IRAK degrader is a Proteolysis Targeting Chimeric (PROTAC).

In some embodiments, the IRAK degrader is an IRAK-1 degrader, an IRAK-2 degrader, an IRAK-M degrader (or IRAK-3 degrader), or an IRAK-4 degrader.

Suitable IRAK4 degrader compounds for use in the methods of the invention are described in patent applications WO2019/133531, WO2020/113233, WO2020/264490, WO 2021/127283, or WO2021/011868.

In some embodiments, the IRAK-4 degrader comprises 54(1R, 4R)-2-oxa-5-azabicyclo[2.2.1]heptan-5-yl)-N-(3-(difluoromethyl)-1((1R, 4R)-4-((4-((3-(1-(2,6-dioxopiperidin-3-yl)-3-methyl-2-oxo-2,3-dihydro-1Hbenzo[d]imidazol-4-yl)prop-2-yn-1-yl)oxy)piperidin-1-yl)methyl)cyclohexyl)-1H-pyrazol-4-yl)pyrazolo[1,5-a]pyrimidine-3-carboxamide and its pharmaceutically acceptable salts, described in WO 2021/247899. In some embodiments, the IRAK-4 degrader comprises a crystalline form of 54(1R, 4R)-2-oxa-5-azabicyclo[2.2.1]heptan-5-yl-N-(3-(difluoromethyl)-1-((1r,4R)-4((4((3-(1-(2,6-dioxopiperidin-3-yl)-3-methyl-2-oxo-2,3-dihydro-1Hbenzo[d]imidazol-4-yl)prop-2-yn-1-yl)oxy)piperidin-1-yl)methyl)cyclohexyl)-1H-pyrazol-4-yl)pyrazolo[1,5-a]pyrimidine-3-carboxamide and its pharmaceutically acceptable salts.

In some embodiments, the IRAK-4 degrader comprises a compound of formula [I] or a deuterated from of the compound of formula [I] or a pharmaceutically acceptable salt thereof (described in WO 2021/247897).

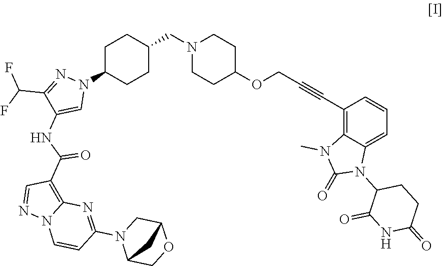 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21]

The compound of formula [I] is 54(1R, 4R)-2-oxa-5-azabicyclo[2.2.1]heptan-5-yl)-N-(3-(difluoromethyl)-1-((1R, 4R)-4-((4-((3-(1-(2,6-dioxopiperidin-3-yl)-3-methyl-2-oxo-2,3-dihydro-1Hbenzo[d]imidazol-4-yl)prop-2-yn-1-yl)oxy)piperidin-1-yl)methyl)cyclohexyl)-1H-pyrazol-4-yl)pyrazolo[1,5-a]pyrimidine-3-carboxamide.

### Gene Therapy Agents

In some aspects, the invention provides methods for using IRAK degraders with gene therapy agents for improved gene therapy by inhibiting an innate immune response to the gene therapy agent. In some embodiments, the gene therapy agent is a viral particle or a lipid nanoparticle. In some embodiments, the gene therapy agent is an adeno-associated virus (AAV) particle, an adenovirus particle, a lentivirus particle, or a herpes simplex virus (HAV) particle. In some embodiments, the gene therapy agent is a lipid nanoparticle or a liposome. In some embodiments, the immune response to the gene therapy agent is an immune response to the viral particle (e.g., viral capsid proteins, viral envelopes, etc.). In some embodiments, the immune response to the gene therapy agent is an immune response to an LNP (e.g., one or more lipids used to produce the LNP). In some embodiments, the immune response to the gene therapy agent is an immune response to the gene therapy payload; e.g., nucleic acid encoding the therapeutic transgene (a viral genome, a plasmid, a closed ended DNA, an mRNA, an antisense nucleic acid, a siRNA, a shRNA and the like). In some embodiments, the immune response to the gene therapy agent is an immune response to the transgene product (e.g., a therapeutic polypeptide or therapeutic nucleic acid).

### AAV

In some embodiments, the invention provides methods for using IRAK degraders with an AAV particle for improved gene therapy. In an AAV particle for gene therapy, a recombinant AAV (rAAV) genome encoding a heterologous nucleic acid (e.g., a therapeutic transgene) is encapsidated in an AAV capsid. In some embodiments, the viral genome comprises a heterologous nucleic acid and/or one or more of the following components, operatively linked in the direction of transcription, control sequences including transcription initiation and termination sequences, thereby forming an expression cassette.

In some embodiments, the rAAV genome comprises one or more AAV inverted terminal repeat (ITR) sequences (typically two AAV ITR sequences). For example, an expression cassette may be flanked on the 5′ and 3′ end by at least one functional AAV ITR sequence. By “functional AAV ITR sequences”, it is meant that the ITR sequences function as intended for the rescue, replication and packaging of the AAV virion. See Davidson et al., PNAS, 2000, 97(7)3428-32; Passini et al., J. Virol., 2003, 77(12):7034-40; and Pechan et al., Gene Ther., 2009, 16:10-16, all of which are incorporated herein in their entirety by reference. For practicing some aspects of the invention, the recombinant viral genomes comprise at least all of the sequences of AAV essential for encapsidation into the AAV capsid and the physical structures for infection by the AAV particle. AAV ITRs for use in the vectors of the invention need not have a wild-type nucleotide sequence (e.g., as described in Kotin, Hum. Gene Ther., 1994, 5:793-801), and may be altered by the insertion, deletion or substitution of nucleotides or the AAV ITRs may be derived from any of several AAV serotypes. More than 40 serotypes of AAV are currently known, and new serotypes and variants of existing serotypes continue to be identified. See Gao et al., PNAS, 2002, 99(18): 11854-6; Gao et al., PNAS, 2003, 100(10):6081-6; and Bossis et al., J. Virol., 2003, 77(12):6799-810. Use of any AAV serotype is considered within the scope of the present invention. In some embodiments, a rAAV vector is a vector derived from an AAV serotype, including without limitation, AAV1, AAV2, AAV3, AAV4, AAV5, AAV6, AAV7, AAV8, AAVrh8, AAVrh8R, AAV9, AAV10, AAVrh10, AAV11, AAV12, AAV LK03, AAV2R471A, AAV DJ, AAV DJ8, a goat AAV, bovine AAV, or mouse AAV ITRs or the like. In some embodiments, the AAV nucleic acid (e.g., an rAAV vector) comprises one or more (e.g., in some aspects two) ITR of AAV1, AAV2, AAV3, AAV4, AAV5, AAV6, AAV7, AAV8, AAVrh8, AAVrh8R, AAV9, AAV10, AAVrh10, AAV11, AAV12, AAV LK03, AAV2R471A, AAV DJ, AAV DJ8, a goat AAV, bovine AAV, or mouse AAV ITRs or the like. In some embodiments, the AAV particle comprises an AAV vector encoding a heterologous transgene flanked by one or more AAV ITRs.

In some embodiments, the AAV particle comprises a capsid protein selected from an AAV1 capsid, an AAV2 capsid, an AAV3 capsid, an AAV4 capsid, an AAV5 capsid, an AAV6 capsid, an AAV7 capsid, an AAV8 capsid, an AAVrh8 capsid, an AAV9 capsid, an AAV10 capsid, an AAVrh10 capsid, an AAV11 capsid, an AAV12 capsid, an AAVrh32.33 capsid, An AAV-XL32 capsid, an AAV-XL32.1 capsid, an AAV LK03 capsid, an AAV2R471A capsid, an AAV2/2-7m8 capsid, an AAV DJ capsid, an AAV DJ8 capsid, an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, a mouse AAV capsid rAAV2/HBoV1 (chimeric AAV/human bocavirus virus 1), an AAV2HBKO capsid, an AAVPHP.B capsid or an AAVPHP.eB capsid, or a functional variant thereof. By “functional variant” of an AAV capsid, it is meant that the variant capsid is capable of packaging an AAV genome to generate an infectious AAV virion. In further embodiments, a rAAV particle comprises capsid proteins of an AAV serotype from Clades A-F.

In some aspects, the invention provides AAV particles comprising a recombinant self-complementing genome (e.g., a self-complementary or self-complimenting AAV vector). AAV viral particles with self-complementing vector genomes and methods of use of self-complementing rAAV genomes are described in U.S. Pat. Nos. 6,596,535; 7,125,717; 7,465,583; 7,785,888; 7,790,154; 7,846,729; 8,093,054; and 8,361,457; and Wang Z., et al., (2003) Gene Ther 10:2105-2111, each of which are incorporated herein by reference in its entirety. An AAV particle comprising a self-complementing genome will quickly form a double stranded DNA molecule by virtue of its partially complementing sequences (e.g., complementing coding and non-coding strands of a heterologous nucleic acid). In some embodiments, the vector comprises a first nucleic acid sequence encoding a heterologous nucleic acid and a second nucleic acid sequence encoding a complement of the nucleic acid, where the first nucleic acid sequence can form intrastrand base pairs with the second nucleic acid sequence along most or all of its length.

In some embodiments, the first heterologous nucleic acid sequence and a second heterologous nucleic acid sequence are linked by a mutated ITR (e.g., the right ITR). In some embodiments, the ITR comprises the polynucleotide sequence 5′-CACTCCCTCTCTGCGCGCTCGCTCGCTCACTGAGGCC GGGCGACCAAAGGTCGCCCACGCCCGGGCTTTGCCCGGGCG-3′ (SEQ ID NO:1). The mutated ITR comprises a deletion of the D region comprising the terminal resolution sequence. As a result, on replicating a rAAV genome, the rep proteins will not cleave the viral genome at the mutated ITR and as such, a recombinant viral genome comprising the following in 5′ to 3′ order will be packaged in a viral capsid: an AAV ITR, the first heterologous polynucleotide sequence including regulatory sequences, the mutated AAV ITR, the second heterologous polynucleotide in reverse orientation to the first heterologous polynucleotide and a third AAV ITR.

Different AAV serotypes are used to optimize transduction of particular target cells or to target specific cell types within a particular target tissue (e.g., a diseased tissue). An AAV particle can comprise viral proteins and viral nucleic acids of the same serotype or a mixed serotype. For example, an AAV particle may contain one or more ITRs and capsid derived from the same AAV serotype, or an AAV particle may contain one or more ITRs derived from a different AAV serotype than capsid of the AAV particle.

In some embodiments, the AAV capsid comprises a mutation, e.g., the capsid comprises a mutant capsid protein. In some embodiments, the mutation is a tyrosine mutation or a heparin binding mutation. In some embodiments, a mutant capsid protein maintains the ability to form an AAV capsid. In some embodiments, the AAV particle comprises an AAV2 or AAV5 tyrosine mutant capsid (see, e.g., Zhong L. et al., (2008) Proc Natl Acad Sci USA 105(22):7827-7832), such as a mutation in Y444 or Y730 (numbering according to AAV2). In further embodiments, the AAV particle comprises capsid proteins of an AAV serotype from Clades A-F (Gao, et al., J. Virol. 2004, 78(12):6381).

Numerous methods are known in the art for production of AAV particles for gene therapy, including transfection, stable cell line production, and infectious hybrid virus production systems which include adenovirus-AAV hybrids, herpesvirus-AAV hybrids (Conway, J E et al., (1997) J. Virology 71(11):8780-8789) and baculovirus-AAV hybrids (Urabe, M. et al., (2002) Human Gene Therapy 13(16):1935-1943; Kotin, R. (2011) Hum Mol Genet. 20(R1): R2-R6). AAV production cultures for the production of AAV particles all require; 1) suitable host cells, 2) suitable helper virus function, 3) AAV rep and cap genes and gene products; 4) a nucleic acid (such as a therapeutic nucleic acid) flanked by at least one AAV ITR sequences; and 5) suitable media and media components to support AAV production. In some embodiments, the suitable host cell is a primate host cell. In some embodiments, the suitable host cell is a human-derived cell lines such as HeLa, A549, 293, or Perc.6 cells. In some embodiments, the suitable helper virus function is provided by wild-type or mutant adenovirus (such as temperature sensitive adenovirus), herpes virus (HSV), baculovirus, or a plasmid construct providing helper functions. In some embodiments, the AAV rep and cap gene products may be from any AAV serotype. In general, but not obligatory, the AAV rep gene product is of the same serotype as the ITRs of the rAAV genome as long as the rep gene products may function to replicated and package the rAAV genome. Suitable media known in the art may be used for the production of AAV particles. In some embodiments, the AAV helper functions are provided by adenovirus or HSV. In some embodiments, the AAV helper functions are provided by baculovirus and the host cell is an insect cell (e.g., Spodoptera frugiperda (Sf9) cells).

One method for producing AAV particles is the triple transfection method. Briefly, a plasmid containing a rep gene and a capsid gene, along with a helper adenoviral plasmid, may be transfected (e.g., using the calcium phosphate method) into a cell line (e.g., HEK-293 cells), and virus may be collected and optionally purified. As such, in some embodiments, the AAV particle was produced by triple transfection of a nucleic acid encoding the AAV vector, a nucleic acid encoding AAV rep and cap, and a nucleic acid encoding AAV helper virus functions into a host cell, wherein the transfection of the nucleic acids to the host cells generates a host cell capable of producing AAV particles.

In some embodiments, AAV particles may be produced by a producer cell line method (see Martin et al., (2013) Human Gene Therapy Methods 24:253-269; U.S. PG Pub. No. US2004/0224411; and Liu, X. L. et al. (1999) Gene Ther. 6:293-299). Briefly, a cell line (e.g., a HeLa, 293, A549, or Perc.6 cell line) may be stably transfected with a plasmid containing a rep gene, a capsid gene, and a vector genome comprising a promoter-heterologous nucleic acid sequence. Cell lines may be screened to select a lead clone for AAV production, which may then be expanded to a production bioreactor and infected with a helper virus (e.g., an adenovirus or HSV) to initiate AAV production. Virus may subsequently be harvested, adenovirus may be inactivated (e.g., by heat) and/or removed, and the AAV particles may be purified. As such, in some embodiments, the AAV particle was produced by a producer cell line comprising one or more of nucleic acid encoding the rAAV genome, a nucleic acid encoding AAV rep and cap, and a nucleic acid encoding AAV helper virus functions.

In some embodiments, the nucleic acid encoding AAV rep and cap genes and/or the AAV viral genome are stably maintained in the producer cell line. In some embodiments, nucleic acid encoding AAV rep and cap genes and/or the rAAV genome is introduced on one or more plasmids into a cell line to generate a producer cell line. In some embodiments, the AAV rep, AAV cap, and AAV genome are introduced into a cell on the same plasmid. In other embodiments, the AAV rep, AAV cap, and rAAV genome are introduced into a cell on different plasmids. In some embodiments, a cell line stably transfected with a plasmid maintains the plasmid for multiple passages of the cell line (e.g., 5, 10, 20, 30, 40, 50 or more than 50 passages of the cell). For example, the plasmid(s) may replicate as the cell replicates, or the plasmid(s) may integrate into the cell genome. A variety of sequences that enable a plasmid to replicate autonomously in a cell (e.g., a human cell) have been identified (see, e.g., Krysan, P. J. et al. (1989) Mol. Cell Biol. 9:1026-1033). In some embodiments, the plasmid(s) may contain a selectable marker (e.g., an antibiotic resistance marker) that allows for selection of cells maintaining the plasmid. Selectable markers commonly used in mammalian cells include without limitation blasticidin, G418, hygromycin B, zeocin, puromycin, and derivatives thereof. Methods for introducing nucleic acids into a cell are known in the art and include without limitation viral transduction, cationic transfection (e.g., using a cationic polymer such as DEAE-dextran or a cationic lipid such as lipofectamine), calcium phosphate transfection, microinjection, particle bombardment, electroporation, and nanoparticle transfection (for more details, see e.g., Kim, T. K. and Eberwine, J. H. (2010) Anal. Bioanal. Chem. 397:3173-3178).

In some embodiments, the producer cell line is derived from a primate cell line (e.g., a non-human primate cell line, such as a Vero or FRhL-2 cell line). In some embodiments, the cell line is derived from a human cell line. In some embodiments, the producer cell line is derived from HeLa, 293, A549, or PERC.6® (Crucell) cells. For example, prior to introduction and/or stable maintenance/integration of nucleic acid encoding AAV rep and cap genes and/or the rAAV genome into a cell line to generate a producer cell line, the cell line is a HeLa, 293, A549, or PERC.6® (Crucell) cell line, or a derivative thereof.

In some embodiments, the producer cell line is adapted for growth in suspension. As is known in the art, anchorage-dependent cells are typically not able to grow in suspension without a substrate, such as microcarrier beads. Adapting a cell line to grow in suspension may include, for example, growing the cell line in a spinner culture with a stirring paddle, using a culture medium that lacks calcium and magnesium ions to prevent clumping (and optionally an antifoaming agent), using a culture vessel coated with a siliconizing compound, and selecting cells in the culture (rather than in large clumps or on the sides of the vessel) at each passage.

AAV particles of the invention may be harvested from AAV production cultures by lysis of the host cells of the production culture or by harvest of the spent media from the production culture, provided the cells are cultured under conditions known in the art to cause release of AAV particles into the media from intact cells, as described more fully in U.S. Pat. No. 6,566,118). Suitable methods of lysing cells are also known in the art and include for example multiple freeze/thaw cycles, sonication, microfluidization, and treatment with chemicals, such as detergents and/or proteases.

In a further embodiment, the AAV particles are purified. The term “purified” as used herein includes a preparation of AAV particles devoid of at least some of the other components that may also be present where the AAV particles naturally occur or are initially prepared from. Thus, for example, isolated AAV particles may be prepared using a purification technique to enrich it from a source mixture, such as a culture lysate or production culture supernatant. Enrichment can be measured in a variety of ways, such as, for example, by the proportion of DNase-resistant particles (DRPs) or genome copies (gc) present in a solution, or by infectivity, or it can be measured in relation to a second, potentially interfering substance present in the source mixture, such as contaminants, including production culture contaminants or in-process contaminants, including helper virus, media components, and the like.

In some embodiments, the AAV production culture harvest is clarified to remove host cell debris. In some embodiments, the production culture harvest is clarified by filtration through a series of depth filters including, for example, a grade DOHC Millipore Millistak+ HC Pod Filter, a grade A1HC Millipore Millistak+ HC Pod Filter, and a 0.2 μm Filter Opticap XL1O Millipore Express SHC Hydrophilic Membrane filter. Clarification can also be achieved by a variety of other standard techniques known in the art, such as, centrifugation or filtration through any cellulose acetate filter of 0.2 μm or greater pore size known in the art.

In some embodiments, the AAV production culture harvest is further treated with Benzonase® to digest any high molecular weight DNA present in the production culture. In some embodiments, the Benzonase® digestion is performed under standard conditions known in the art including, for example, a final concentration of 1-2.5 units/ml of Benzonase® at a temperature ranging from ambient to 37° C. for a period of 30 minutes to several hours.

AAV particles may be isolated or purified using one or more of the following purification steps: equilibrium centrifugation; flow-through anionic exchange filtration; tangential flow filtration (TFF) for concentrating the AAV particles; AAV capture by apatite chromatography; heat inactivation of helper virus; AAV capture by hydrophobic interaction chromatography; buffer exchange by size exclusion chromatography (SEC); nanofiltration; and AAV capture by anionic exchange chromatography, cationic exchange chromatography, or affinity chromatography. These steps may be used alone, in various combinations, or in different orders. In some embodiments, the method comprises all the steps in the order as described below. Methods to purify AAV particles are found, for example, in Xiao et al., (1998) Journal of Virology 72:2224-2232; U.S. Pat. Nos. 6,989,264 and 8,137,948; and WO 2010/148143.

### Adenovirus

In some embodiments, the invention provides methods for using IRAK degraders with an adenovirus particle for improved gene therapy. Adenoviral vectors for gene therapy are typically adenoviral particles with a recombinant adenovirus (rAd) genome comprising one or more heterologous sequences (i.e., nucleic acid sequence not of adenoviral origin) between two adenoviral ITRs encapsidated into an adenoviral capsid. In some embodiments, the heterologous sequence encodes a therapeutic transgene. In some embodiments, the rAd genome lacks or contains a defective copy of one or more E1 genes, which renders the adenovirus replication-defective. Adenoviruses include a linear, double-stranded DNA genome within a large (˜950Å), non-enveloped icosahedral capsid. Adenoviruses have a large genome that can incorporate more than 30 kb of heterologous sequence (e.g., in place of the E1 and/or E3 region), making them uniquely suited for use with larger heterologous genes. They are also known to infect dividing and non-dividing cells and do not naturally integrate into the host genome (although hybrid variants may possess this ability). In some embodiments, the adenoviral vector may be a first generation adenoviral vector with a heterologous sequence in place of E1. In some embodiments, the adenoviral vector may be a second generation adenoviral vector with additional mutations or deletions in E2A, E2B, and/or E4. In some embodiments, the adenoviral vector may be a third generation or gutted adenoviral vector that lacks all viral coding genes, retaining only the ITRs and packaging signal and requiring a helper adenovirus in trans for replication, and packaging. Adenoviral particles have been investigated for use as vectors for transient transfection of mammalian cells as well as gene therapy vectors. For further description, see, e.g., Danthinne, X. and Imperiale, M. J. (2000) Gene Ther. 7:1707-14 and Tatsis, N. and Ertl, H. C. (2004) Mol. Ther. 10:616-29.

In some embodiments, the adenoviral particle comprises a rAd genome comprising a therapeutic transgene. Use of any adenovirus serotype is considered within the scope of the present invention. In some embodiments, the adenoviral particle is derived from an adenovirus serotype, including without limitation, AdHu2, AdHu 3, AdHu4, AdHu5, AdHu7, AdHu11, AdHu24, AdHu26, AdHu34, AdHu35, AdHu36, AdHu37, AdHu41, AdHu48, AdHu49, AdHu50, AdC6, AdC7, AdC69, bovine Ad type 3, canine Ad type 2, ovine Ad, and porcine Ad type 3. The adenoviral particle also comprises capsid proteins. In some embodiments, the adenoviral particle includes one or more foreign viral capsid proteins. Such combinations may be referred to as pseudotyped adenoviral particles. In some embodiments, foreign viral capsid proteins used in pseudotyped adenoviral particles are derived from a foreign virus or from another adenovirus serotype. In some embodiments, the foreign viral capsid proteins are derived from, including without limitation, reovirus type 3. Examples of vector and capsid protein combinations used in pseudotyped adenovirus particles can be found in the following references (Tatsis, N. et al. (2004) Mol. Ther. 10(4):616-629 and Ahi, Y. et al. (2011) Curr. Gene Ther. 11(4):307-320). Different adenovirus serotypes can be used to optimize transduction of particular target cells or to target specific cell types within a particular target tissue (e.g., a diseased tissue). Tissues or cells targeted by specific adenovirus serotypes, include without limitation, lung (e.g. HuAd3), spleen and liver (e.g. HuAd37), smooth muscle, synoviocytes, dendritic cells, cardiovascular cells, tumor cell lines (e.g. HuAd11), and dendritic cells (e.g. HuAd5 pseudotyped with reovirus type 3, HuAd30, or HuAd35). For further description, see Ahi, Y. et al. (2011) Curr. Gene Ther. 11(4):307-320, Kay, M. et al. (2001) Nat. Med. 7(1):33-40, and Tatsis, N. et al. (2004) Mol. Ther. 10(4):616-629.

Numerous methods are known in the art for production of adenoviral particles. For example, for a gutted adenoviral vector, the adenoviral vector genome and a helper adenovirus genome may be transfected into a packaging cell line (e.g., a 293 cell line). In some embodiments, the helper adenovirus genome may contain recombination sites flanking its packaging signal, and both genomes may be transfected into a packaging cell line that expresses a recombinase (e.g., the Cre/loxP system may be used), such that the adenoviral vector of interest is packaged more efficiently than the helper adenovirus (see, e.g., Alba, R. et al. (2005) Gene Ther. 12 Suppl 1:S18-27). Adenoviral vectors may be harvested and purified using standard methods, such as those described herein.

### Lentivirus

In some embodiments, the invention provides methods for using IRAK degraders with a lentivirus particle for improved gene therapy. Lentiviral vectors for gene therapy are typically lentiviral particles with a recombinant lentivirus genome comprising one or more heterologous sequences (i.e., nucleic acid sequence not of lentiviral origin) between two long terminal repeats (LTRs). In some embodiments, the heterologous sequence encodes a therapeutic transgene. Lentiviruses are positive-sense, ssRNA retroviruses with a genome of approximately 10 kb. Lentiviruses integrate into the genome of dividing and non-dividing cells. Lentiviral particles may be produced, for example, by transfecting multiple plasmids (typically the lentiviral genome and the genes required for replication and/or packaging are separated to prevent viral replication) into a packaging cell line, which packages the modified lentiviral genome into lentiviral particles. In some embodiments, a lentiviral particle may refer to a first generation vector that lacks the envelope protein. In some embodiments, a lentiviral particle may refer to a second-generation vector that lacks all genes except the gag/pol and tat/rev regions. In some embodiments, a lentiviral particle may refer to a third generation vector that only contains the endogenous rev, gag, and pol genes and has a chimeric LTR for transduction without the tat gene (see Dull, T. et al. (1998) J. Virol. 72:8463-71). For further description, see Durand, S. and Cimarelli, A. (2011) Viruses 3:132-59.

Use of any lentiviral vector is considered within the scope of the present invention. In some embodiments, the lentiviral vector is derived from a lentivirus including, without limitation, human immunodeficiency virus-1 (HIV-1), human immunodeficiency virus-2 (HIV-2), simian immunodeficiency virus (SIV), feline immunodeficiency virus (FIV), equine infectious anemia virus (EIAV), bovine immunodeficiency virus (BIV), Jembrana disease virus (JDV), visna virus (VV), and caprine arthritis encephalitis virus (CAEV). The lentiviral particle also comprises capsid proteins. In some embodiments, the lentivirus particles include one or more foreign viral capsid proteins. Such combinations may be referred to as pseudotyped lentiviral particles. In some embodiments, foreign viral capsid proteins used in pseudotyped lentiviral particles are derived from a foreign virus. In some embodiments, the foreign viral capsid protein used in pseudotyped lentiviral particles is Vesicular stomatitis virus glycoprotein (VSV-GP). VSV-GP interacts with a ubiquitous cell receptor, providing broad tissue tropism to pseudotyped lentiviral particles. In addition, VSV-GP is thought to provide higher stability to pseudotyped lentiviral particles. In other embodiments, the foreign viral capsid proteins are derived from, including without limitation, Chandipura virus, Rabies virus, Mokola virus, Lymphocytic choriomeningitis virus (LCMV), Ross River virus (RRV), Sindbis virus, Semliki Forest virus (SFV), Venezuelan equine encephalitis virus, Ebola virus Reston, Ebola virus Zaire, Marburg virus, Lassa virus, Avian leukosis virus (ALV), Jaagsiekte sheep retrovirus (JSRV), Moloney Murine leukemia virus (MLV), Gibbon ape leukemia virus (GALV), Feline endogenous retrovirus (RD114), Human T-lymphotropic virus 1 (HTLV-1), Human foamy virus, Maedi-visna virus (MVV), SARS-CoV, Sendai virus, Respiratory syncytia virus (RSV), Human parainfluenza virus type 3, Hepatitis C virus (HCV), Influenza virus, Fowl plague virus (FPV), or Autographa californica multiple nucleopolyhedro virus (AcMNPV). Examples of vector and capsid protein combinations used in pseudotyped lentivirus particles can be found, for example, in Cronin, J. et al. (2005). Curr. Gene Ther. 5(4):387-398. Different pseudotyped lentiviral particles can be used to optimize transduction of particular target cells or to target specific cell types within a particular target tissue (e.g., a diseased tissue). For example, tissues targeted by specific pseudotyped lentiviral particles, include without limitation, liver (e.g. pseudotyped with a VSV-G, LCMV, RRV, or SeV F protein), lung (e.g. pseudotyped with an Ebola, Marburg, SeV F and HN, or JSRV protein), pancreatic islet cells (e.g. pseudotyped with an LCMV protein), central nervous system (e.g. pseudotyped with a VSV-G, LCMV, Rabies, or Mokola protein), retina (e.g. pseudotyped with a VSV-G or Mokola protein), monocytes or muscle (e.g. pseudotyped with a Mokola or Ebola protein), hematopoietic system (e.g. pseudotyped with an RD114 or GALV protein), or cancer cells (e.g. pseudotyped with a GALV or LCMV protein). For further description, see Cronin, J. et al. (2005). Curr. Gene Ther. 5(4):387-398 and Kay, M. et al. (2001) Nat. Med. 7(1):33-40.

Numerous methods are known in the art for production of lentiviral particles. For example, for a third-generation lentiviral vector, a vector containing the recombinant lentiviral genome of interest with gag and pol genes may be co-transfected into a packaging cell line (e.g., a 293 cell line) along with a vector containing a rev gene. The recombinant lentiviral genome of interest also contains a chimeric LTR that promotes transcription in the absence of Tat (see Dull, T. et al. (1998) J. Virol. 72:8463-71). Lentiviral vectors may be harvested and purified using methods (e.g., Segura M M, et al., (2013) Expert Opin Biol Ther. 13(7):987-1011) described herein.

### HSV

In some embodiments, the invention provides methods for using IRAK degraders with a HSV particle for improved gene therapy. HSV vectors for gene therapy are typically HSV particles with a recombinant HSV genome comprising one or more heterologous sequences (i.e., nucleic acid sequence not of HSV origin) between two terminal repeats (TRs). In some embodiments, the heterologous sequence encodes a therapeutic transgene. HSV is an enveloped, double-stranded DNA virus with a genome of approximately 152 kb. Advantageously, approximately half of its genes are nonessential and may be deleted to accommodate heterologous sequence. HSV particles infect non-dividing cells. In addition, they naturally establish latency in neurons, travel by retrograde transport, and can be transferred across synapses, making them advantageous for transfection of neurons and/or gene therapy approaches involving the nervous system. In some embodiments, the HSV particle may be replication-defective or replication-competent (e.g., competent for a single replication cycle through inactivation of one or more late genes). For further description, see Manservigi, R. et al. (2010) Open Virol. J. 4:123-56.

In some embodiments, the HSV particle comprising a recombinant HSV genome comprising a transgene. Use of any HSV vector is considered within the scope of the present invention. In some embodiments, the HSV vector is derived from a HSV serotype, including without limitation, HSV-1 and HSV-2. The HSV particle also comprises capsid proteins. In some embodiments, the HSV particles include one or more foreign viral capsid proteins. Such combinations may be referred to as pseudotyped HSV particles. In some embodiments, foreign viral capsid proteins used in pseudotyped HSV particles are derived from a foreign virus or from another HSV serotype. In some embodiments, the foreign viral capsid protein used in a pseudotyped HSV particle is a Vesicular stomatitis virus glycoprotein (VSV-GP). VSV-GP interacts with a ubiquitous cell receptor, providing broad tissue tropism to pseudotyped HSV particles. In addition, VSV-GP is thought to provide higher stability to pseudotyped HSV particles. In other embodiments, the foreign viral capsid protein may be from a different HSV serotype. For example, an HSV-1 vector may contain one or more HSV-2 capsid proteins. Different HSV serotypes can be used to optimize transduction of particular target cells or to target specific cell types within a particular target tissue (e.g., a diseased tissue). Tissues or cells targeted by specific adenovirus serotypes include without limitation, central nervous system and neurons (e.g. HSV-1). For further description, see Manservigi, R. et al. (2010) Open Virol J 4:123-156, Kay, M. et al. (2001) Nat. Med. 7(1):33-40, and Meignier, B. et al. (1987) J. Infect. Dis. 155(5):921-930.

Numerous methods are known in the art for production of HSV particles. HSV vectors may be harvested and purified using standard methods, such as those described herein. For example, for a replication-defective HSV vector, an HSV genome of interest that lacks all of the immediate early (IE) genes may be transfected into a complementing cell line that provides genes required for virus production, such as ICP4, ICP27, and ICP0 (see, e.g., Samaniego, L. A. et al. (1998) J. Virol. 72:3307-20). HSV vectors may be harvested and purified using methods described (e.g., Goins, W F et al., (2014) Herpes Simplex Virus Methods in Molecular Biology 1144:63-79).

### Nonviral Gene Therapy Agents

In some embodiments, the invention provides methods for using IRAK degraders with non-viral gene transfer methods for gene therapy. Non-viral vector delivery systems include DNA plasmids, naked nucleic acid, and nucleic acid complexed to a delivery system. For example, the vector may be complexed to a lipid (e.g., a cationic or neutral lipid), a liposome, a polycation, a lipid nanoparticle, or an agent that enhances the cellular uptake of nucleic acid. The nucleic acid may be complexed to an agent suitable for any of the delivery methods described herein. In some embodiments, the nucleic acid encodes a therapeutic transgene.

Lipid nanoparticles for gene therapy typically comprise a vector genome encapsulated in a lipid particle or a vector genome complexed with a lipid. In some embodiments, the heterologous sequence encodes a therapeutic transgene. In some embodiments, the vector genome is formulated in a lipoplex nanoparticle or liposome. In some embodiments, a lipoplex nanoparticle formulation for the gene therapy agent comprises the synthetic cationic lipid (R)—N,N,N-trimethyl-2,3-dioleyloxy-1-propanaminium chloride (DOTMA) and the phospholipid 1,2-dioleoyl-sn-glycero-3-phosphoethanolamine (DOPE). In some embodiments, the DOTMA/DOPE liposomal component is optimized for delivery and targeting of cells in the individual.

In some embodiments, nucleic acid comprising the vector genome is mixed with a pharmaceutical composition comprising one or more cationic lipids, including, e.g., (R)—N,N,N-trimethyl-2,3-dioleyloxy-1-propanaminium chloride (DOTMA) and the phospholipid 1,2-dioleoyl-sn-glycero-3-phosphoethanolamine (DOPE). In some embodiments, the pharmaceutical composition comprises at least one lipid. In some embodiments, the pharmaceutical composition comprises at least one cationic lipid. The cationic lipid can be monocationic or polycationic. Any cationic amphiphilic molecule, e.g., a molecule which comprises at least one hydrophilic and lipophilic moiety is a cationic lipid within the meaning of the present invention. In some embodiments, the positive charges are contributed by the at least one cationic lipid and the negative charges are contributed by the nucleic acid. In some embodiments, the pharmaceutical composition comprises at least one helper lipid. The helper lipid may be a neutral or an anionic lipid. The helper lipid may be a natural lipid, such as a phospholipid or an analogue of a natural lipid, or a fully synthetic lipid, or lipid-like molecule, with no similarities with natural lipids. In one embodiment, the cationic lipid and/or the helper lipid is a bilayer forming lipid. Examples of helper lipids include, but are not limited to, 1,2-di-(9Z-octadecenoyl)-sn-glycero-3-phosphoethanolamine (DOPE) or analogs or derivatives thereof, cholesterol (Chol) or analogs or derivatives thereof and/or 1,2-dioleoyl-sn-glycero-3-phosphocholine (DOPC) or analogs or derivatives thereof.

In some embodiments, the molar ratio of the at least one cationic lipid to the at least one helper lipid is from 10:0 to 3:7, preferably 9:1 to 3:7, 4:1 to 1:2, 4:1 to 2:3, 7:3 to 1:1, or 2:1 to 1:1, preferably about 1:1. In some embodiments, in this ratio, the molar amount of the cationic lipid results from the molar amount of the cationic lipid multiplied by the number of positive charges in the cationic lipid.

In some embodiments, the lipid is comprised in a vesicle encapsulating the vector genome. The vesicle may be a multilamellar vesicle, an unilamellar vesicle, or a mixture thereof. The vesicle may be a liposome.

### Vector Genomes

In some embodiments, the invention provides methods for using IRAK degraders with a gene therapy agent for the delivery of a therapeutic transgene to a desired target in the individual. In some embodiments, the gene therapy agent comprises a vector genome for delivery and expression of the therapeutic transgene in the desired target in the individual.

The present invention contemplates the use of gene therapy agents for the introduction of one or more nucleic acid sequences encoding a therapeutic polypeptide and/or nucleic acid for packaging into a viral particle (for viral gene therapy agents). The vector genome may include any element to establish the expression of the therapeutic polypeptide and/or nucleic acid, for example, a promoter, an ITR of the present disclosure, a ribosome binding element, terminator, enhancer, selection marker, intron, polyA signal, and/or origin of replication.

In some embodiments, the therapeutic transgene encodes a therapeutic polypeptide. A therapeutic polypeptide may, e.g., supply a polypeptide and/or enzymatic activity that is absent or present at a reduced level in a cell or organism. Alternatively, a therapeutic polypeptide may supply a polypeptide and/or enzymatic activity that indirectly counteracts an imbalance in a cell or organism. For example, a therapeutic polypeptide for a disorder related to buildup of a metabolite caused by a deficiency in a metabolic enzyme or activity may supply a missing metabolic enzyme or activity, or it may supply an alternate metabolic enzyme or activity that leads to reduction of the metabolite. A therapeutic polypeptide may also be used to reduce the activity of a polypeptide (e.g., one that is overexpressed, activated by a gain-of-function mutation, or whose activity is otherwise misregulated) by acting, e.g., as a dominant-negative polypeptide.

The vector genomes of the invention may encode polypeptides that are intracellular proteins, anchored in the cell membrane, remain within the cell, or are secreted by the cell transduced with the vectors of the invention. For polypeptides secreted by the cell that receives the vector; the polypeptide can be soluble (i.e., not attached to the cell). For example, soluble polypeptides are devoid of a transmembrane region and are secreted from the cell. Techniques to identify and remove nucleic acid sequences which encode transmembrane domains are known in the art.

In some embodiments, the vector genome of the invention encodes polypeptides used to treat a disease or disorder in an individual. Diseases and disorders treated by the gene therapy agent of the invention include but are not limited to Huntington disease (HD), progressive supranuclear palsy (PSP), multiple system atrophy (MSA), metachromatic leukodystrophy (MLD), amyotrophic lateral sclerosis (ALS), age-related macular degeneration (AMD), congenital muscular dystrophy (CMD), phenylketonuria (PKU), muscular dystrophy (MD), A1AT deficiency, focal segmental glomerulosclerosis (FSGS), cystinuria, hemophilia A, hemophilia B, Gaucher disease (GBA), Parkinson's disease (PD), and Pompe disease.

In some embodiments, the therapeutic polypeptide is huntingtin (HTT), tau, amyloid precursor protein, alpha-synuclein, pseudoarylsulfatase (ARSA), superoxide dismutase 1 (SOD1), phenylalanine hydroxylase (PAH), dystrophin, alpha-1-antitrypsin (A1AT), cysteine transporter, Factor VIII (FVIII), Factor IX (FIX), acid beta-glucosidase, glial-derived growth factor (GDNF), brain-derived growth factor (BDNF), tyrosine hydroxlase (TH), GTP-cyclohydrolase (GTPCH), and/or amino acid decarboxylase (AADC), or alpha glucosidase.

In some embodiments, the heterologous nucleic acid encodes a therapeutic nucleic acid e.g. that can be used to replace, or knock down, one or more defective genes. In some embodiments, a therapeutic nucleic acid may include without limitation an DNA, siRNA, an shRNA, an RNAi, a miRNA, an antisense RNA, a ribozyme or a DNAzyme. As such, a therapeutic nucleic acid may encode an RNA that when transcribed from the nucleic acids of the vector can treat a disorder by interfering with translation or transcription of an abnormal or excess protein associated with a disorder of the invention. For example, the nucleic acids of the invention may encode for an RNA which treats a disorder by highly specific elimination or reduction of mRNA encoding the abnormal and/or excess proteins. Therapeutic RNA sequences include RNAi, small inhibitory RNA (siRNA), micro RNA (miRNA), and/or ribozymes (such as hammerhead and hairpin ribozymes) that can treat disorders by highly specific elimination or reduction of mRNA encoding the abnormal and/or excess proteins.

In some embodiments, the therapeutic polypeptide or therapeutic nucleic acid is used to treat a disorder of the CNS. Without wishing to be bound to theory, it is thought that a therapeutic polypeptide or therapeutic nucleic acid may be used to replace a mutated gene with a wild type or improved gene, reduce or eliminate the expression and/or activity of a polypeptide whose gain-of-function has been associated with a disorder, or to enhance the expression and/or activity of a polypeptide to complement a deficiency that has been associated with a disorder (e.g., a mutation in a gene whose expression shows similar or related activity). Non-limiting examples of disorders of the invention that may be treated by a therapeutic polypeptide or therapeutic nucleic acid of the invention (exemplary genes that may be targeted or supplied are provided in parenthesis for each disorder) include stroke (e.g., caspase-3, Beclin1, Ask1, PAR1, HIF1α, PUMA, and/or any of the genes described in Fukuda, A. M. and Badaut, J. (2013) Genes (Basel) 4:435-456), Huntington's disease (mutant HTT), epilepsy (e.g., SCN1A, NMDAR, ADK, and/or any of the genes described in Boison, D. (2010) Epilepsia 51:1659-1668), Parkinson's disease (alpha-synuclein), Lou Gehrig's disease (also known as amyotrophic lateral sclerosis; SOD1), Alzheimer's disease (tau, amyloid precursor protein), corticobasal degeneration or CBD (tau), corticogasal ganglionic degeneration or CBGD (tau), frontotemporal dementia or FTD (tau), progressive supranuclear palsy or PSP (tau), multiple system atrophy or MSA (alpha-synuclein), cancer of the brain (e.g., a mutant or overexpressed oncogene implicated in brain cancer), and lysosomal storage diseases (LSD). Disorders of the invention may include those that involve large areas of the cortex, e.g., more than one functional area of the cortex, more than one lobe of the cortex, and/or the entire cortex. Other non-limiting examples of disorders of the invention that may be treated by a therapeutic polypeptide or therapeutic nucleic acid of the invention include traumatic brain injury, enzymatic dysfunction disorders, psychiatric disorders (including post-traumatic stress syndrome), neurodegenerative diseases, and cognitive disorders (including dementias, autism, and depression). Enzymatic dysfunction disorders include without limitation leukodystrophies (including Canavan's disease) and any of the lysosomal storage diseases described below.

In some embodiments, the therapeutic polypeptide or therapeutic nucleic acid is used to treat a lysosomal storage disease. As is commonly known in the art, lysosomal storage disease are rare, inherited metabolic disorders characterized by defects in lysosomal function. Such disorders are often caused by a deficiency in an enzyme required for proper mucopolysaccharide, glycoprotein, and/or lipid metabolism, leading to a pathological accumulation of lysosomally stored cellular materials. Non-limiting examples of lysosomal storage diseases of the invention that may be treated by a therapeutic polypeptide or therapeutic nucleic acid of the invention (exemplary genes that may be targeted or supplied are provided in parenthesis for each disorder) include Gaucher disease type 2 or type 3 (acid beta-glucosidase, GBA), GM1 gangliosidosis (beta-galactosidase-1, GLB1), Hunter disease (iduronate 2-sulfatase, IDS), Krabbe disease (galactosylceramidase, GALC), a mannosidosis disease (a mannosidase, such as alpha-D-mannosidase, MAN2B1), β mannosidosis disease (beta-mannosidase, MANBA), metachromatic leukodystrophy disease (pseudoarylsulfatase A, ARSA), mucolipidosisII/III disease (N-acetylglucosamine-1-phosphotransferase, GNPTAB), Niemann-Pick A disease (acid sphingomyelinase, ASM), Niemann-Pick C disease (Niemann-Pick C protein, NPC1), Pompe disease (acid alpha-1,4-glucosidase, GAA), Sandhoff disease (hexosaminidase beta subunit, HEXB), Sanfilippo A disease (N-sulfoglucosamine sulfohydrolase, MPS3A), Sanfilippo B disease (N-alpha-acetylglucosaminidase, NAGLU), Sanfilippo C disease (heparin acetyl-CoA:alpha-glucosaminidase N-acetyltransferase, MPS3C), Sanfilippo D disease (N-acetylglucosamine-6-sulfatase, GNS), Schindler disease (alpha-N-acetylgalactosaminidase, NAGA), Sly disease (beta-glucuronidase, GUSB), Tay-Sachs disease (hexosaminidase alpha subunit, HEXA), and Wolman disease (lysosomal acid lipase, LIPA).

In some embodiments, the therapeutic polypeptide encodes Factor VIII, Factor IX, myotubularin, survival motor neuron protein (SMN), retinoid isomerohydrolase (RPE65), NADH-ubiquinone oxidoreductase chain 4, Choroideremia protein (CHM), ornithine transcarbomylase, argininosuccinate synthetase, β-globin, γ-globin, phenylalanine hydroxylase, adrenoleukodystrophy protein (ALD), dystrophin, a truncated dystrophin, an anti-VEGF agent, or a functional variant thereof.

In some embodiments, the heterologous nucleic acid is operably linked to a promoter. Exemplary promoters include, but are not limited to, the cytomegalovirus (CMV) immediate early promoter, the RSV LTR, the MoMLV LTR, the phosphoglycerate kinase-1 (PGK) promoter, a simian virus 40 (SV40) promoter and a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), the E2F promoter, the telomerase (hTERT) promoter; the cytomegalovirus enhancer/chicken beta-actin/Rabbit β-globin promoter (CAG promoter; Niwa et al., Gene, 1991, 108(2):193-9) and the elongation factor 1-alpha promoter (EF1-alpha) promoter (Kim et al., Gene, 1990, 91(2):217-23 and Guo et al., Gene Ther., 1996, 3(9):802-10). In some embodiments, the promoter comprises a human (3-glucuronidase promoter or a cytomegalovirus enhancer linked to a chicken β-actin (CBA) promoter. The promoter can be a constitutive, inducible or repressible promoter. In some embodiments, the invention provides a recombinant vector comprising nucleic acid encoding a heterologous transgene of the present disclosure operably linked to a CBA promoter. Exemplary promoters and descriptions may be found, e.g., in U.S. PG Pub. 20140335054.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the 13-actin promoter, the phosphoglycerol kinase (PGK) promoter, and the EFla promoter [Invitrogen].

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter, or fragment thereof, for the transgene will be used. The native promoter can be used when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art.

In some embodiments, the vector comprises an intron. For example, in some embodiments, the intron is a chimeric intron derived from chicken beta-actin and rabbit beta-globin. In some embodiments, the intron is a minute virus of mice (MVM) intron.

In some embodiments, the vector comprises a polyadenylation (polyA) sequence. Numerous examples of polyadenylation sequences are known in the art, such as a bovine growth hormone (BGH) Poly(A) sequence (see, e.g., accession number EF592533), an SV40 polyadenylation sequence, and an HSV TK pA polyadenylation sequence.

Methods for Selecting a Patient for Treatment with a Gene Therapy Agent and an IRAK Degrader

In some aspects, the invention provides methods for delivering a nucleic acid to a cell of an individual, the method comprising a) incubating innate immune cells from the individual with the gene therapy agent (e.g., an AAV particle, an adenovirus particle, a lentivirus particle, a HSV particle, or a lipid nanoparticle), b) analyzing the innate immune cells for the expression of one or more cytokines wherein expression of a cytokine signature following incubation with the gene therapy agent identifies an individual with innate immunity to the gene therapy agent, c) administering an IRAK degrader (e.g., an IRAK-4 degrader) to the individual identified in step b), and d) administering the gene therapy agent to the individual identified in step b). In some embodiments, the innate immune cells are dendritic cells, monocytes, macrophages, or natural killer (NK) cells. In some embodiments, the method further includes the steps of isolating innate immune cells from the individual prior to incubating the innate immune cells with the gene therapy agent. In some embodiments, the method further includes the steps of isolating monocytes from the individual and, incubating the monocytes in dendritic cell culture media to derive dendritic cells from the monocytes prior to incubating the dendritic cells with the gene therapy agent.

In some aspects, the invention provides methods for treating an individual in need thereof, the method comprising a) incubating innate immune cells with the gene therapy agent (e.g., an AAV particle, an adenovirus particle, a lentivirus particle, a HSV particle, or a lipid nanoparticle), b) analyzing the innate immune cells for the expression of one or more cytokines wherein expression of a cytokine signature following incubation with the gene therapy agent identifies an individual with innate immunity to the gene therapy agent, c) administering an IRAK degrader (e.g., an IRAK-4 degrader) to the individual identified in step b), and d) administering the gene therapy agent to the individual identified in step b).). In some embodiments, the innate immune cells are dendritic cells, monocytes, macrophages, or natural killer (NK) cells. In some embodiments, the method further includes the steps of isolating innate immune cells from the individual prior to incubating the innate immune cells with the gene therapy agent. In some embodiments, the method further includes the steps of isolating monocytes from the individual and, incubating the monocytes in dendritic cell culture media to derive dendritic cells from the monocytes prior to incubating the dendritic cells with the gene therapy agent.

In some aspects, the invention provides methods for selecting an individual for treatment with a gene therapy agent (e.g., an AAV particle, an adenovirus particle, a lentivirus particle, a HSV particle, or a lipid nanoparticle) and an IRAK degrader (e.g., an IRAK-4 degrader), the method comprising a) incubating innate immune cells with the gene therapy agent, b) analyzing the innate immune cells for the expression of one or more cytokines, wherein expression of a cytokine signature following incubation with the gene therapy agent identifies an individual for treatment with a gene therapy agent and an IRAK degrader, c) selecting the individual identified in step b) for treatment with a gene therapy agent and an IRAK degrader. In some embodiments, the methods further comprise the steps of d) administering an IRAK degrader to the individual identified in step b), and e) administering the gene therapy agent to the individual identified in step b). In some embodiments, the innate immune cells are dendritic cells, monocytes, macrophages, or natural killer (NK) cells. In some embodiments, the method further includes the steps of isolating innate immune cells from the individual prior to incubating the innate immune cells with the gene therapy agent. In some embodiments, the method further includes the steps of isolating monocytes from the individual and, incubating the monocytes in dendritic cell culture media to derive dendritic cells from the monocytes prior to incubating the dendritic cells with the gene therapy agent.

In some embodiments, the innate immune cells are isolated from peripheral blood mononuclear cells from the individual. In some embodiments, the innate immune cell is a dendritic cell. In some embodiments, the dendritic cell is derived (e.g., differentiated) from a monocyte of the individual. In some embodiments, the monocytes are isolated from peripheral blood mononuclear cells from the individual. In some embodiments, the monocytes are CD14+ monocytes. In some embodiments, the monocytes are incubated with the dendritic cell culture media for about 5 to about 10 days or about 7 to about 8 days to derive dendritic cells from the monocytes. In some embodiments, the monocytes are incubated with the dendritic cell culture media for about any of 3, 4, 5, 6, 7, 8, 9, 10, 11, 12, or more than 12 days to derive dendritic cells from the monocytes. In some embodiments, the dendritic cells are replated prior to the incubation with the gene therapy agent of step c). In some embodiments, the dendritic cells are replated into microwell dishes prior to incubation with the gene therapy agent.

In some embodiments, the dendritic cells are incubated with the viral gene therapy agent at an MOI of about 1×10³ to about 1×10⁵ or about 1×10⁴. In some embodiments, the dendritic cells are incubated with the gene therapy agent at an MOI of less than about any of 1×10³, 5×10³, 1×10⁴, 5×10⁴, 1×10⁵, or 5×10⁵.

In some embodiments, the dendritic cells are incubated with a non-viral gene therapy agent at a concentration of about 1 ng/mL to about 1 mg/mL. In some embodiments, the dendritic cells are incubated with the non-viral gene therapy agent at a concentration of about 1 ng/mL to about 10 ng/mL, about 10 ng/mL to about 100 ng/mL, about 100 ng/mL to about 1 μg/mL, about 1 μg/mL to about 10 μg/mL, about 10 μg/mL to about 100 μg/mL, or about 100 μg/mL to about 1 mg/mL.

In some embodiments, the dendritic cells are incubated with the gene therapy agent for about 12 hours to about 36 hours or about 24 hours. In some embodiments, the dendritic cells are incubated with the gene therapy agent for between about 6 hours and about 48 hours, about 6 hours and about 36 hours, about 6 hours and about 24 hours, about 6 hours and about 18 hours, about 6 hours and about 12 hours, about 12 hours and about 48 hours, about 12 hours and about 36 hours, about 12 hours and about 24 hours, about 12 hours and about 18 hours, about 18 hours and about 48 hours, about 18 hours and about 36 hours, about 18 hours and about 24 hours, about 24 hours and about 48 hours, about 24 hours and about 36 hours, or about 36 hours and about 48 hours.

In some embodiments, a cytokine signature is determined for a gene therapy agent in a particular immune cell (e.g., a dendritic cell, a monocyte, a macrophage, an NK cell, etc.) by contacting the particular immune cells from a plurality of individuals with a gene therapy agent and determining changes in expression of one or more cytokines associated with an innate immune response, wherein a commonality in changes in expression (e.g., increased or decreased expression) in the one or more cytokines indicates the presence of a cytokine signature. In some embodiments, the cytokines associated with an innate immune response are associated with a toll-like receptor (TLR) pathway (e.g., a TLR2, TLR3, TLR4 or TLR9 pathway). In some embodiments, the cytokine signature comprises changes in expression in more than any of 1, 2, 3, 4, 5, 6, 7, 8, 9, or 10 cytokines. In some embodiments, the plurality of individuals comprises more than any of 1, 2, 3, 4, 5, 6, 7, 8, 9, or 10 individuals. In some embodiments, the commonality of changes in expression comprises similar changes in expression levels of cytokines in innate immune cells in greater than about 25%, 50%, 75% or 90% of the individuals in the plurality of individuals.

In some embodiments, the cytokine signature comprises increased expression of one or more of IL6, TNFα, IL-1β, MCP1 and MIP-1α. In some embodiments, the cytokine signature comprises increased expression of two or more of IL6, TNFα, IL-1β, MCP1 and MIP-1α. In some embodiments, the cytokine signature comprises increased expression of three or more of IL6, TNFα, IL-1β, MCP1 and MIP-1α. In some embodiments, the cytokine signature comprises increased expression of four or more of IL6, TNFα, IL-1β, MCP1 and MIP-1α. In some embodiments, the cytokine signature comprises increased expression of IL6, TNFα, IL-1β, MCP1 and MIP-1α. In some embodiments, the cytokine signature comprises increased expression of IL6, TNFα, and IL-1β.

In some embodiments, the innate immune cell is a dendritic cell and the cytokine signature comprises increased expression of one or more of IL6, TNFα, IL-1β, MCP1 and MIP-1α. In some embodiments, the innate immune cell is a dendritic cell and the cytokine signature comprises increased expression of two or more of IL6, TNFα, IL-1β, MCP1 and MIP-1α. In some embodiments, the innate immune cell is a dendritic cell and the cytokine signature comprises increased expression of three or more of IL6, TNFα, IL-1β, MCP1 and MIP-1α. In some embodiments, the cytokine signature comprises increased expression of four or more of IL6, TNFα, IL-1β, MCP1 and MIP-1α. In some embodiments, the innate immune cell is a dendritic cell and the cytokine signature comprises increased expression of IL6, TNFα, IL-1β, MCP1 and MIP-1α. In some embodiments, the cytokine signature comprises increased expression of IL6, TNFα, and IL-1β.

In some embodiments, expression of the cytokines in the cytokine signature is increased compared to expression of the cytokines in a suitable control. Examples of a suitable control include the cytokine signature from innate immune cells that are not incubated with (in the absence of) the gene therapy agent and expression of the cytokines in the cytokine signature from the same or similar innate immune cells prior to incubation with the gene therapy agent (e.g., wherein the cytokine signature comprises increased expression of one or more of IL6, TNFα, IL-1β, MCP1 and MIP-1α). In some embodiments, the cytokine signature comprises increased expression of two or more of IL6, TNFα, IL-1β, MCP1 and MIP-1α. In some embodiments, the cytokine signature comprises increased expression of three or more of IL6, TNFα, IL-1β, MCP1 and MIP-1α. In some embodiments, the cytokine signature comprises increased expression of four or more of IL6, TNFα, IL-1β, MCP1 and MIP-1α. In some embodiments, the cytokine signature comprises increased expression of IL6, TNFα, IL-1β, MCP1 and MIP-1α. In some embodiments, the cytokine signature comprises increased expression of IL6, TNFα, and IL-1β. In some embodiments, an increase in expression of any one of about 10%, about 20%, about 25%, about 50%, about 75%, about 100%, or more than 100% identifies an individual for treatment with a gene therapy agent and an IRAK degrader.

In some embodiments, expression of the cytokines in the cytokine signature is increased compared to expression of the cytokines in the cytokine signature from dendritic cells incubated in the absence of the gene therapy agent or compared to expression of the cytokines in the cytokine signature from dendritic cells prior to incubation with the gene therapy agent, wherein the cytokine signature comprises increased expression of one or more of IL6, TNFα, IL-1β, MCP1 and MIP-1α. In some embodiments, the cytokine signature comprises increased expression of two or more of IL6, TNFα, IL-1β, MCP1 and MIP-1α. In some embodiments, the cytokine signature comprises increased expression of three or more of IL6, TNFα, IL-1β, MCP1 and MIP-1α. In some embodiments, the cytokine signature comprises increased expression of four or more of IL6, TNFα, IL-1β, MCP1 and MIP-1α. In some embodiments, the cytokine signature comprises increased expression of IL6, TNFα, IL-1β, MCP1 and MIP-1α. In some embodiments, the cytokine signature comprises increased expression of IL6, TNFα, and IL-1β. In some embodiments, an increase in expression of any one of about 10%, about 20%, about 25%, about 50%, about 75%, about 100%, or more than 100% identifies an individual for treatment with a gene therapy agent and an IRAK degrader.

Pharmaceutical compositions

In some aspects, the invention is directed a pharmaceutical composition comprising the gene therapy agent (e.g., an AAV particle, an adenovirus particle, a lentivirus particle, a HSV particle, or a lipid nanoparticle) and/or the IRAK degrader (e.g., an IRAK-4 degrader) as described herein. The pharmaceutical compositions may be suitable for any mode of administration described herein or known in the art.

In some embodiments, the pharmaceutical composition comprising a pharmaceutically acceptable excipient. As is well known in the art, pharmaceutically acceptable excipients are relatively inert substances that facilitate administration of a pharmacologically effective substance and can be supplied as liquid solutions or suspensions, as emulsions, or as solid forms suitable for dissolution or suspension in liquid prior to use. For example, an excipient can give form or consistency, or act as a diluent. Suitable excipients include but are not limited to stabilizing agents, wetting and emulsifying agents, salts for varying osmolarity, encapsulating agents, pH buffering substances, and buffers. Such excipients include any pharmaceutical agent suitable for direct delivery to the eye which may be administered without undue toxicity. Pharmaceutically acceptable excipients include, but are not limited to, sorbitol, any of the various TWEEN compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in REMINGTON'S PHARMACEUTICAL SCIENCES (Mack Pub. Co., N.J. 1991). In some embodiments, the pharmaceutical composition comprising a rAAV particle described herein and a pharmaceutically acceptable carrier is suitable for administration to human. Such carriers are well known in the art (see, e.g., Remington's Pharmaceutical Sciences, 15th Edition, pp. 1035-1038 and 1570-1580).

Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. The pharmaceutical composition may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms. The compositions are generally formulated as sterile and substantially isotonic solution.

### Kits and Articles of Manufacture

The gene therapy agent (e.g., an AAV particle, an adenovirus particle, a lentivirus particle, a HSV particle, or a lipid nanoparticle) and/or the IRAK degrader (e.g., an IRAK-4 degrader) as described herein may be contained within a kit or article of manufacture, e.g., designed for use in one of the methods of the invention as described herein.

In some embodiments, the kits or articles of manufacture further include instructions for administration of the IRAK degrader and/or gene therapy agent. The kits or articles of manufacture described herein may further include other materials desirable from a commercial and user standpoint, including other buffers, diluents, filters, needles, syringes, and package inserts with instructions for performing any methods described herein. Suitable packaging materials may also be included and may be any packaging materials known in the art, including, for example, vials (such as sealed vials), vessels, ampules, bottles, jars, flexible packaging (e.g., sealed Mylar or plastic bags), and the like. These articles of manufacture may further be sterilized and/or sealed.

In some embodiments, the kits or articles of manufacture further contain one or more of the buffers and/or pharmaceutically acceptable excipients described herein (e.g., as described in REMINGTON'S PHARMACEUTICAL SCIENCES (Mack Pub. Co., N.J. 1991). In some embodiments, the kits or articles of manufacture include one or more pharmaceutically acceptable excipients, carriers, solutions, and/or additional ingredients described herein. The kits or articles of manufacture described herein can be packaged in single unit dosages or in multidosage forms. The contents of the kits or articles of manufacture are generally formulated as sterile and can be lyophilized or provided as a substantially isotonic solution.

### Exemplary Embodiments

The invention includes the following enumerated embodiments.

1. A method for delivering nucleic acid to a cell of an individual, the method comprising a) administering an IRAK degrader to the individual, and b) administering a gene therapy agent to the individual.

2. A method for treating an individual in need thereof with a gene therapy agent, the method comprising a) administering an IRAK degrader to the individual, and b) administering the gene therapy agent to the individual.

3. A method for improving gene therapy in an individual, the method comprising a) administering an IRAK degrader to the individual, and b) administering a gene therapy agent to the individual.

4. A method for suppressing an immune response to a gene therapy agent, the method comprising a) administering an IRAK degrader to the individual, and b) administering a gene therapy agent to the individual.

5. The method of any one of embodiments 1-5, wherein the IRAK degrader modulates the activity or expression of an IRAK protein kinase.

6. The method of embodiment 5, wherein the IRAK protein kinase is an IRAK-1 protein kinase, an IRAK-2 protein kinase, an IRAK-3 protein kinase, or an IRAK-4 protein kinase.

7. The method of any one of embodiments 1-6, wherein the IRAK degrader modulates the activity or expression of an IRAK-4 protein kinase.

8. The method of any one of embodiments 1-10, wherein the IRAK degrader comprises the compound of formula [I]:

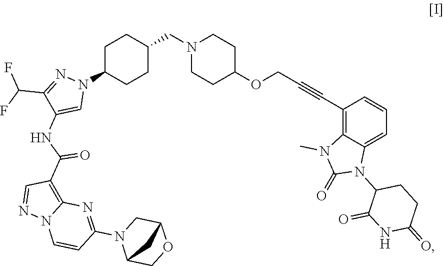 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21]

or a pharmaceutically acceptable salt thereof.

9. The method of any one of embodiments 1-8, wherein the gene therapy agent comprises a viral vector.

10. The method of embodiment 9, wherein the viral vector is an AAV particle.

11. The method of embodiment 10, wherein the AAV particle comprises an AAV1 capsid, an AAV2 capsid, an AAV3 capsid, an AAV4 capsid, an AAV5 capsid, an AAV6 capsid, an AAV7 capsid, an AAV8 capsid, an AAVrh8 capsid, an AAV9 capsid, an AAV10 capsid, an AAVrh10 capsid, an AAV11 capsid, an AAV12 capsid, an AAVrh32.33 capsid, An AAV-XL32 capsid, an AAV-XL32.1 capsid, an AAV LK03 capsid, an AAV2R471A capsid, an AAV2/2-7m8 capsid, an AAV DJ capsid, an AAV DJ8 capsid, an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, a mouse AAV capsid rAAV2/HBoV1 (chimeric AAV/human bocavirus virus 1), an AAV2HBKO capsid, an AAVPHP.B capsid or an AAVPHP.eB capsid, or a functional variant thereof.

12. The method of embodiment 11, wherein the AAV capsid comprises a tyrosine mutation, a heparin binding mutation, or an HBKO mutation.

13. The method of any one of embodiments 10-12, wherein the AAV viral particle comprises an AAV genome comprising one or more inverted terminal repeats (ITRs), wherein the one or more ITRs is an AAV1 ITR, an AAV2 ITR, an AAV3 ITR, an AAV4 ITR, an AAV5 ITR, an AAV6 ITR, an AAV7 ITR, an AAV8 ITR, an AAVrh8 ITR, an AAV9 ITR, an AAV10 ITR, an AAVrh10 ITR, an AAV11 ITR, or an AAV12 ITR.

14. The method of embodiment 13, wherein the one or more ITRs and the capsid of the AAV particle are derived from the same AAV serotype.

15. The method of embodiment 13, wherein the one or more ITRs and the capsid of the AAV particles are derived from different AAV serotypes.

16. The method of embodiment 9, where viral vector is an adenoviral particle.

17. The method of embodiment 16, wherein the adenoviral particle comprises a capsid from Adenovirus serotype 2, 1, 5, 6, 19, 3, 11, 7, 14, 16, 21, 12, 18, 31, 8, 9, 10, 13, 15, 17, 19, 20, 22, 23, 24-30, 37, 40, 41, AdHu2, AdHu 3, AdHu4, AdHu24, AdHu26, AdHu34, AdHu35, AdHu36, AdHu37, AdHu41, AdHu48, AdHu49, AdHu50, AdC6, AdC7, AdC69, bovine Ad type 3, canine Ad type 2, ovine Ad, or porcine Ad type 3, or a functional variant thereof.

18. The method of embodiment 9, where the viral vector is a lentiviral particle.

19. The method of embodiment 18, wherein the lentiviral particle is pseudotyped with vesicular stomatitis virus (VSV), lymphocytic choriomeningitis virus (LCMV), Ross river virus (RRV), Ebola virus, Marburg virus, Mokala virus, Rabies virus, RD114, or a functional variant thereof.

20. The method of embodiment 9, where the viral vector is a Herpes simplex virus (HSV) particle.

21. The method of embodiment 20, wherein the HSV particle is an HSV-1 particle or an HSV-2 particle, or a functional variant thereof.

22. The method of any one of embodiments 1-8, wherein the gene therapy agent comprises a lipid nanoparticle.

23. The method of any one of embodiments 1-22, wherein the gene therapy agent comprises nucleic acid encoding a heterologous transgene.

24. The method of embodiment 23, wherein the heterologous transgene is operably linked to a promoter.

25. The method of embodiment 24, wherein the promoter is a constitutive promoter, a tissue-specific promoter, or an inducible promoter.

26. The method of any one of embodiments 1-25, wherein the IRAK degrader is administered before, at the same time, or after administration of the gene therapy agent.

27. The method of any one of embodiments 1-26, wherein the individual has a disease or disorder suitable for treatment by gene therapy.

28. The method of embodiment 27, wherein the disease or disorder is a monogenic disease or disorder.

29. The method of any one of embodiments 1-28, wherein the gene therapy agent is administered intravenously, intraperitoneally, intra-arterially, intramuscularly, subcutaneously, or intrahepatically.

30. The method of any one of embodiments 1-29, wherein the IRAK degrader is administered orally, intravenously, intraperitoneally, intra-arterially, intramuscularly, subcutaneously, or intrahepatically.

31. A method for delivering a nucleic acid to a cell of an individual, the method comprising a) incubating innate immune cells from the individual with the gene therapy agent, b) analyzing the innate immune cells for the expression of one or more cytokines wherein expression of a cytokine signature following incubation with the gene therapy agent identifies an individual with innate immunity to the gene therapy agent, c) administering an IRAK degrader to the individual identified in step b), and d) administering the gene therapy agent to the individual identified in step b).

32. A method for treating an individual in need thereof, the method comprising a) incubating innate immune cells from the individual with the gene therapy agent, b) analyzing the innate immune cells for the expression of one or more cytokines wherein expression of a cytokine signature following incubation with the gene therapy agent identifies an individual with innate immunity to the gene therapy agent, c) administering an IRAK degrader to the individual identified in step b), and d) administering the gene therapy agent to the individual identified in step b).

33. A method for selecting an individual for treatment with a gene therapy agent and an IRAK degrader, the method comprising a) incubating innate immune cells from the individual with the gene therapy agent, b) analyzing the innate immune cells for the expression of one or more cytokines wherein expression of a cytokine signature following incubation with the gene therapy agent identifies an individual for treatment with a gene therapy agent and an IRAK degrader, c) selecting the individual identified in step b) for treatment with a gene therapy agent and an IRAK degrader.

34. The method of any one of embodiments 31-33, wherein the innate immune cell is a dendritic cell, a monocyte, a macrophage or a natural killer (NK) cell.

35. The method of any one of embodiments 31-34, wherein the innate immune cells are isolated from peripheral blood mononuclear cells from the individual.

36. The method of any one of embodiments 31-35, wherein the innate immune cell is a dendritic cell.

37. The method of embodiment 36, wherein the dendritic cell is derived from a monocyte of the individual.

38. The method of embodiment 37 further comprising isolating monocytes from the individual and incubating the monocytes in dendritic cell culture media to derive dendritic cells from the monocytes prior to incubating the dendritic cells with the gene therapy agent.

39. The method of embodiment 37 or 38, wherein the monocytes are CD14+ monocytes.

40. The method of any one of embodiments 37-39, wherein the monocytes are incubates with the dendritic cell culture media for about 5 to about 10 days or about 7 to about 8 days to derive dendritic cells from the monocytes.

41. The method of any one of embodiments 31-40, wherein the dendritic cells are replated prior to the incubation with the gene therapy agent of step c).

42. The method of embodiment 41, wherein the dendritic cells are replated into microwell dishes.

43. The method of any one of embodiments 31-42, wherein the gene therapy agent is a viral vector, and wherein the innate immune cells are incubated with the viral vector at an MOI of about 1×103 to about 1×105 or about 1×104.

44. The method of any one of embodiments 31-42, wherein the gene therapy agent is a non-viral vector, and wherein the innate immune cells are incubated with the non-viral vector at a concentration of about 1 ng/mL to about 1 mg/mL.

45. The method of any one of embodiments 31-44, wherein the innate immune cells are incubated with the gene therapy agent for about 12 hours to about 36 hours or about 24 hours.

46. The method of any one of embodiments 31-45, wherein the cytokine signature comprises increased expression of one or more of IL6, TNFα, IL-1β, MCP1 and MIP-1 a.

47. The method of any one of embodiments 31-46, wherein the cytokine signature comprises increased expression of IL6, TNFα, IL-1β, MCP1 and MIP-1a.

48. The method of any one of embodiments 31-47, wherein the cytokine signature comprises increased expression of IL6, TNFα, and IL-1β.

49. The method of any one of embodiments 331-48, wherein expression of the cytokines in the cytokine signature is increased compared to a suitable control.

50. The method of embodiment 49, wherein the suitable control is the expression of the cytokines in the cytokine signature from innate immune cells that are not incubated with the gene therapy agent or wherein the suitable control is expression of the cytokines in the cytokine signature from innate immune cells prior to incubation with the gene therapy agent.

51. The method of any one of embodiments 31-50, wherein the IRAK degrader modulates the activity of an IRAK protein kinase.

52. The method of embodiment 51, wherein the IRAK protein kinase is an IRAK-1 protein kinase, an IRAK-2 protein kinase, an IRAK-3 protein kinase, or an IRAK-4 protein kinase.

53. The method of any one of embodiments 31-52, wherein the IRAK degrader modulates the activity of an IRAK-4 protein kinase.

54. The method of any one of embodiments 31-52, wherein the IRAK degrader is a small molecule.

55. The method of any one of embodiments 31-54, wherein the IRAK degrader comprises the compound of formula [I]:

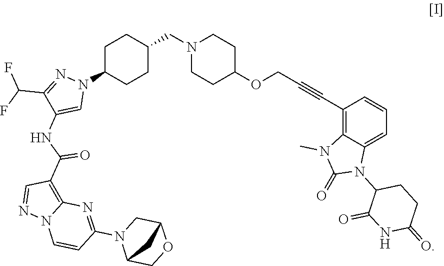 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21]

or a pharmaceutically acceptable salt thereof.

56. The method of any one of embodiments 31-55, wherein the IRAK degrader blocks TLR9 function.

57. The method of any one of embodiments 31-56, wherein the gene therapy agent is a viral vector.

58. The method of embodiment 57, wherein the viral vector is an AAV particle.

59. The method of embodiment 58, wherein the AAV particle comprises an AAV1 capsid, an AAV2 capsid, an AAV3 capsid, an AAV4 capsid, an AAV5 capsid, an AAV6 capsid, an AAV7 capsid, an AAV8 capsid, an AAVrh8 capsid, an AAV9 capsid, an AAV10 capsid, an AAVrh10 capsid, an AAV11 capsid, an AAV12 capsid, an AAVrh32.33 capsid, An AAV-XL32 capsid, an AAV-XL32.1 capsid, an AAV LK03 capsid, an AAV2R471A capsid, an AAV2/2-7m8 capsid, an AAV DJ capsid, an AAV DJ8 capsid, an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, a mouse AAV capsid rAAV2/HBoV1 (chimeric AAV/human bocavirus virus 1), an AAV2HBKO capsid, an AAVPHP.B capsid or an AAVPHP.eB capsid, or a functional variant thereof.

60. The method of embodiment 59, wherein the AAV capsid comprises a tyrosine mutation, a heparin binding mutation, or an HBKO mutation.

61. The method of any one of embodiments 58-60, wherein the AAV viral particle comprises an AAV genome comprising one or more inverted terminal repeats (ITRs), wherein the one or more ITRs is an AAV1 ITR, an AAV2 ITR, an AAV3 ITR, an AAV4 ITR, an AAV5 ITR, an AAV6 ITR, an AAV7 ITR, an AAV8 ITR, an AAVrh8 ITR, an AAV9 ITR, an AAV10 ITR, an AAVrh10 ITR, an AAV11 ITR, or an AAV12 ITR.

62. The method of embodiment 61, wherein the one or more ITRs and the capsid of the AAV particle are derived from the same AAV serotype.

63. The method of embodiment 61, wherein the one or more ITRs and the capsid of the AAV particles are derived from different AAV serotypes.

64. The method of embodiment 57, where viral vector is an adenoviral particle.

65. The method of embodiment 64, wherein the adenoviral particle comprises an capsid from Adenovirus serotype 2, 1, 5, 6, 19, 3, 11, 7, 14, 16, 21, 12, 18, 31, 8, 9, 10, 13, 15, 17, 19, 20, 22, 23, 24-30, 37, 40, 41, AdHu2, AdHu 3, AdHu4, AdHu24, AdHu26, AdHu34, AdHu35, AdHu36, AdHu37, AdHu41, AdHu48, AdHu49, AdHu50, AdC6, AdC7, AdC69, bovine Ad type 3, canine Ad type 2, ovine Ad, or porcine Ad type 3, or a functional variant thereof.

66. The method of embodiment 57, where the viral vector is a lentiviral particle.

67. The method of embodiment 66, wherein the recombinant lentiviral particle is pseudotyped with vesicular stomatitis virus (VSV), lymphocytic choriomeningitis virus (LCMV), Ross river virus (RRV), Ebola virus, Marburg virus, Mokala virus, Rabies virus, RD114, or a functional variant thereof.

68. The method of embodiment 57, where the viral vector is a Herpes simplex virus (HSV) particle.

69. The method of embodiment 68, wherein the HSV particle is an HSV-1 particle or an HSV-2 particle, or a functional variant thereof.

70. The method of any one of embodiments 31-565, wherein the gene therapy agent is a lipid nanoparticle.

71. The method of any one of embodiments 31-70, wherein the gene therapy agent comprises nucleic acid encoding a heterologous transgene.

72. The method of embodiment 71, wherein the heterologous transgene is operably linked to a promoter.

73. The method of embodiment 72, wherein the promoter is a constitutive promoter, a tissue-specific promoter, or an inducible promoter.

74. The method of any one of embodiments 31-73, wherein the IRAK degrader is administered before, at the same time, or after administration of the gene therapy agent.

75. The method of any one of embodiments 31-74, wherein the individual has a disease or disorder suitable for treatment by gene therapy.

76. The method of embodiment 75, wherein the disease or disorder is a monogenic disease or disorder.

77. The method of any one of embodiments 31-76, wherein the gene therapy agent is administered intravenously, intraperitoneally, intra-arterially, intramuscularly, subcutaneously, or intrahepatically.

78. The method of any one of embodiments 31-77, wherein the IRAK degrader is administered orally, intravenously, intraperitoneally, intra-arterially, intramuscularly, subcutaneously, or intrahepatically.

79. Use of a composition in the manufacture of a medicament for delivering nucleic acid to a cell of an individual in need thereof, wherein the composition comprises a gene therapy agent, and wherein the composition is formulated for use in combination with an IRAK degrader.

80. Use of a composition in the manufacture of a medicament for delivering nucleic acid to a cell of an individual in need thereof, wherein the composition comprises an IRAK degrader, and wherein the composition is formulated for use in combination with a gene therapy agent.

81. Use of a composition in the manufacture of a medicament for treating an individual in need of gene therapy, wherein the composition comprises a gene therapy agent, and wherein the composition is formulated for use in combination with an IRAK degrader.

82. Use of a composition in the manufacture of a medicament for treating an individual in need of gene therapy, wherein the composition comprises an IRAK degrader, and wherein the composition is formulated for use in combination with a gene therapy agent.

83. Use of a composition in the manufacture of a medicament for modulating an immune response to gene therapy in an individual in need of gene therapy, wherein the composition comprises a gene therapy agent, and wherein the composition is formulated for use in combination with an IRAK degrader.

84. Use of a composition in the manufacture of a medicament for modulating an immune response to gene therapy in an individual, wherein the composition comprises an IRAK degrader, and wherein the composition is formulated for use in combination with a gene therapy agent.

85. The use of any one of embodiments 79-84, wherein the gene therapy agent is an AAV particle, an adenovirus particle, a lentivirus particle, a HSV particle, or a lipid nanoparticle.

86. The use of any one of embodiments 79-85, wherein the IRAK degrader is an IRAK-4 degrader.

87. A composition comprising a gene therapy agent for use in delivering nucleic acid to a cell of an individual in need thereof, wherein the gene therapy agent is used in combination with an IRAK degrader.

88. A composition comprising an IRAK degrader for use in delivering nucleic acid to a cell of an individual in need thereof, wherein the IRAK degrader is used in combination with a gene therapy agent.

89. A composition comprising a gene therapy agent for use in treating an individual in need of gene therapy, wherein the gene therapy agent is used in combination with an IRAK degrader.

90. A composition comprising an IRAK degrader for use in treating an individual in need of gene therapy, wherein the IRAK degrader is used in combination with a gene therapy agent.

91. A composition comprising an IRAK degrader for modulating an immune response to gene therapy in an individual in need of gene therapy, wherein the IRAK degrader is used in combination with a gene therapy agent.

92. A composition comprising an IRAK degrader for suppressing an immune response to gene therapy in an individual in need of gene therapy, wherein the IRAK degrader is used in combination with a gene therapy agent.

93. The composition of any one of embodiments 87-92, wherein the gene therapy agent is an AAV particle, an adenovirus particle, a lentivirus particle, a HSV particle, or a lipid nanoparticle.

94. The composition of any one of embodiments 87-93, wherein the IRAK degrader is an IRAK-4 degrader.

95. A kit for use in the method of any one of embodiments 1-78.

96. A kit for the use of any one of embodiments 78-86.

97. A kit comprising the composition of any one of embodiments 87-94

### Examples

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Ex Vivo Protocol to Assess the Effect of IRAK4 Degraders on Human Cells Treated with AAVs

This example provides a strategy to examine the effects of IRAK4 inhibition on lymphocytes and dendritic cells exposed to AAV.

AAVs trigger an immune response involving activation of both the innate and adaptive immune systems. While the adaptive immune response to AAV is relatively well characterized, innate immune activation by AAV is poorly understood.

IRAK4 is a kinase within the TLR pathway, which activates the innate immune response. While broad acting immune suppressants improve AAV delivery, this result in loss of transgene expression, side effects, and risk of opportunistic infection. Inhibiting IRAk4 may thus result in a more specific immune modulation favorable for AAV treatment.

To prepare peripheral blood mononuclear cells (PBMCs), blood from leukopaks is decanted into 50 mL tubes and DPBS added at a 1:1 ratio. Blood is gently pipetted into a separate 50 mL tube containing 15 mL Ficoll (GE17-5442-02) to ensure no mixing of the blood and Ficoll layers. The mixture is centrifuged at 2000 RPM for 25 minutes at room temperature (9 acceleration and no braking). The buffy coat containing white blood cells and platelets is collected, transferred to a new tube, and centrifuged for five minutes at 400 RCF. Cells are washed three times with PBS containing 1% FBS or FCS and counted.

CD14+ monocytes are isolated from PBMCs using CD14 MicroBeads following the manufacturer's protocol available online (world wide web miltenyibiotec.com/upload/assets/IM0001260.PDF; Miltenyi Biotech, Germany, order no. 130-050-201). Briefly, cells are incubated with 20 μL of CD14 MicroBeads per 10⁷ total cells for 15 minutes at 2-8° C. Cells are applied onto a magnetic column and unlabeled cells are allowed to pass through. After washing the column three times, the column is removed from the magnetic separator, placed on a collection tube, and magnetically labeled cells are flushed out by pushing a plunger into the column.

Monocytes are differentiated into dendritic cells using ImmunoCult-ACF Dendritic Cell Media, Differentiation Supplement, and Maturation Supplement (cdn.stemcell.com/media/Files/pis/DX20521-PIS_1_2_0.pdf?_ga=2.81451927.1035383195.16421057001174975582.1603298321; Stem Cell Technologies, catalog #10986, #10988, and #10989) following the manufacturer's protocol available online. Briefly, purified monocytes are added to dendritic cell media containing the differentiation supplement and incubated at 37° C. for three days. On day 3, media is replaced and cells are incubated for two additional days. Maturation supplement is added on day five at a 1 to 100 dilution (e.g. 50 μL supplement per 5 mL culture).

Differentiated cells are harvested on day seven. T cells are stimulated by adding 4 mL ImmunoCult XF T Cell expansion media (Stem Cell Technologies, catalog #10981) to thawed PBMCs. Cells are spun at 400 g for 5 minutes, media is aspirated and cells are rested overnight in 10 mL of fresh expansion media.

After collection and counting, rested cells are spun at 400 g for five minutes and resuspended. Cell stimulation is performed in a 96 well plate in T cell complete medium. The final concentration of cytokines in the medium are 100 IU IL-2, 5 ng/mL IL-7, and 25 ng/mL IL-15. AAV stimulation is performed at 1e5 MOI, with 10 μg PHA-p used as a positive control. Hemi-depletion is performed every three days. Ten days after thawing PBMCs, cells are stimulated again with AAVs in plain T cell media. Media is collected following 24 hour stimulation.

rAAV vectors (e.g., AAV1, AAV2, and/or AAVrh32.33) are produced using a standard triple transfection method (Sena-Esteves and Gao, Cold Spring Harb Protoc; doi:10.1101/pdb.top095513, 2020). Viruses are purified by cesium chloride ultracentrifugation and titrated using both silver staining and quantitative polymerase chain reaction (qPCR).

Lymphocytes and dendritic cells are incubated with AAV vectors and are either pre-treated, post-treated, or co-treated with an IRAK4 degrader. Pre- and post-treatment with IRAK4 degraders is performed at 3, 6, 12, 24, or 48 hours before or after AAV incubation. Cells are incubated with AAVs at 1e5 MOI for 6, 12, 24, 48, or 72 hours. Cells are treated with the compound of formula [I] (Kymera Therapeutics) at a dose ranging from 1 nM to 1M. LPS (300 ng/mL for 24 hours, Sigma L2630100MG) and R848 (1 μg/mL for 24 hours, Invitrogen tlrl-r948-5) are used as controls. Additional test groups include treatment with either AAV alone, an IRAK4 inhibitor alone, or an IRAK4 inhibitor with LPS or R848.

To determine the extent the TLR9 pathway is inhibited in cells treated with an IRAK4 degrader, cell media is collected and analyzed using Meso Scale Discovery (MSD) assays to determine levels of cytokine release. The V-PLEX Human Biomarker 54-Plex Kit is used and assays are performed according to the manufacturer's protocols (Meso Scale Discovery, Rockville, MD).

### Example 2: In Vivo Analysis of IRAK4 Degraders in Mice Treated with AAVs or LNPs

This example provides an in vivo strategy to examine whether AAV immunogenicity is dampened when animals are treated with an IRAK4 degrader.

All animal housekeeping, maintenance, treatments, and experiments are performed based on Institutional Animal Care and Use Committee (IACUC) guidelines.

Animals receive either AAV or LNP injections, and are either pre-treated, post-treated, or co-treated with an IRAK4 inhibitor. Pre- and post-treatment with IRAK4 degraders is performed at 6 hours, overnight, 24, or 48 hours before or after AAV or LNP injection. Intramuscular AAV injections are performed at a dose of 5×10¹¹ vg/kg and monitored for 7, 14, 21, 28, 35, 42, and 63 days. LNP injections are either intravenous, subcutaneous, or intraperitoneal at doses of 1 mg/kg, 10 mg/kg, or 100 mg/kg. In addition, animals are either pre-treated, post-treated, or co-treated with 1 mg/kg, 10 mg/kg, or 100 mg/kg of the compound of formula [I] (Kymera Therapeutics) given subcutaneously, intraperitoneally, or orally. Additional test groups include treatment with either AAV alone, an IRAK4 inhibitor alone, or an IRAK4 inhibitor with LPS or R848.

Animals are sacrificed 7, 14, 21, 28, 35, 42, or 63 days following treatment and muscle, liver, bone marrow, spleen, blood, and thymus are isolated. Tissue samples are immediately frozen on dry ice. DNA and RNA samples are extracted and vector genome copies and transgene expression are quantified using real-time quantitative PCR (qPCR).

Tissue inflammation in treated animals is assayed using hematoxylin and eosin (H&E) staining. Staining is performed on muscle and liver tissues that are formalin fixed and paraffin embedded according to methods well known in the art. (Gernoux, G et al., Mol Ther, 2020, 28(3):747-757).

Immunohistochemistry is performed on muscle, liver, and spleen cryosections following methods well known in the art (Gernoux, G et al., Mol Ther, 2020, 28(3):747-757). Samples are collected on slides, air dried, and fixed with 3% paraformaldehyde. Samples are analyzed for activated immune cells, such as T cells (using anti-CD3, CD4, and MHC II) and macrophages (using anti-F4/80 and MHC II) and AAV transgene expression. Nuclear and cytoplasmic β-gal is assessed using standard X-gal protocols.

To analyze cytokine levels in treated animals, serum from these subjects is collected and used for Meso Scale Discovery (MSD) assays. The V-PLEX Human Biomarker 54-Plex Kit is used and assays are performed according to the manufacturer's protocol (Meso Scale Discovery, Rockville, MD).

To measure IFN-γ secretion in treated animals, ELISpot assays are performed on splenocytes isolated following injection (Gernoux, G et al., Mol Ther, 2020, 28(3):747-757). Isolated cells are stimulated in vitro for 48 hours using AAVs. Spot number is determined using an iSpot ELISpot Reader ELR068IFL (AID) and analyzed using AID ELISpot Reader software v.6.0. Responses are considered positive with spot-forming units exceed 50 per 1e6 cells and are at least three-fold higher than in control conditions. Unstimulated cells are used as a negative control and CEFT and CD3/CD28 stimulation are used as positive controls.

Single cell suspensions are obtained from spleen and bone marrow samples. Cells are incubated in CD16/32 (Fc Block; BD Biosciences) and stained with antibodies. Cells are acquired on a LSR II (BD Biosciences) and analyzed with FlowJo software (Tree Star).

To analyze cytokine expression in treated animals, isolated splenocytes are stained for IFN-γ, TNF-α, IL-2, IL-4, and IL-6 using intracellular cytokine staining. Splenocytes are prepared as described in Mays, J Immunol 2009 182(1) 6051-6060. Splenocytes are plated in T cell assay medium supplemented with 1 μg/mL brefeldin A (GolgiPlug, BD Pharmigen) and 20 ng/mL mouse IL-2 (BD Pharmingen). Before staining, cells are stimulated with AAVs in the presence or absence of an IRAK4 degrader for 5 hours at 37° C. in 10% CO2. After stimulation, cells are washed, stained with antibodies, and examined using flow cytometry. Samples are acquired on an LSR II and analyzed using FlowJo software.

The T cell response of animals treated with AAV and IRAK4 degraders is determined by MHC class I tetramer staining using PE-conjugated MHC class I H2-Kb-ICPMYARV tetramer complex (Beckman Coulter) following the protocol described in Mays, J Immunol 2009 182(1) 6051-6060. At various times following treatment with AAV and IRAK4 degraders, tetramer staining is performed on heparinized whole blood cells isolated by retro-orbital bleeds. Cells are co-stained for 30 minutes at room temperature with PE-conjugated tetramer and FITC-conjugated anti-CD8a (Ly-2) antibodies (BD Pharmingen) and fixed with iTAg MHC tetramer lysing solution supplemented with fix solution (Beckman Coulter) for 15 minutes at room temperature. Cells are washed three times in PBS and resuspended in 0.01% BD CytoFix (BD Biosciences). Samples are acquired on an LSR II and analyzed using FlowJo software.

### Example 3: Ex Vivo Analysis of the Effect of IRAK4 Degraders KT-474 on Human Cells Treated with AAVs

### Materials and Methods

Preparation of Peripheral Blood Mononuclear Cells. Blood from leukopaks (Stem cell technologies) from different donors was decanted into 50 mL tubes and Dulbecco's phosphate buffered saline (DPBS) was added at a 1:1 ratio. The blood plus DBPS mix was gently pipetted into a separate 50 mL tube containing 15 mL Ficoll (GE17-5442-02) to ensure no mixing of the blood and Ficoll phases. The mixture was centrifuged at 2000 RPM for 25 minutes at room temperature (9 acceleration and no braking). The buffy coat containing the peripheral blood mononuclear cells (PBMCs) was collected, transferred to a new tube, and centrifuged for five minutes at 400 RCF. PBMCs were washed three times with phosphate buffered saline (PBS) containing 1% fetal bovine serum (FBS) or fetal calf serum (FCS) and counted.

Isolation of Monocytes. CD14+ monocytes were isolated from the PBMCs using CD14 MicroBeads following the manufacturer's protocol (Miltenyi Biotech, Germany, order no. 130-050-201, protocol available online world wide web miltenyibiotec.com/upload/assets/IM0001260.PDF). Briefly, PBMCs were incubated with 20 of CD14 MicroBeads per 10⁷ total cells for 15 minutes at 2-8° C. PBMCs were applied onto a magnetic column (Miltenyi; world wide web miltenyibiotec.com/US-en/products/ls-columns.html #130-042-401) and unlabeled cells were allowed to pass through. After washing the column three times, the column was removed from the magnetic separator, (Miltneyi; world wide web miltenyibiotec.com/US-en/products/quadromacs-separator-and-starting-kits.html #130-091-051) placed on a collection tube, and magnetically labeled CD14+ monocytes were flushed out by pushing a plunger into the column.

Differentiation of Monocytes. CD14+ monocytes were differentiated into dendritic cells using ImmunoCult-ACF Dendritic Cell Media, Differentiation Supplement, and Maturation Supplement (Stem Cell Technologies, catalog #10986, #10988, and #10989; world wide web cdn.stemcell.com/media/Files/pis/DX20521-PIS_1_2_0.pdf?_ga=2.81451927.1035383195.1642105700-1174975582.1603298321) following the manufacturer's protocol available online. Briefly, purified CD14+ monocytes were added to Dendritic Cell Media containing the Differentiation Supplement and incubated at 37° C. for three days. On day 3, media was replaced with fresh Dendritic Cell media containing Differentiation Supplement and cells were incubated for two additional days. On day 5, Maturation Supplement was added to the cells at a 1 to 100 dilution (e.g., 50 μL supplement per 5 mL culture). Differentiated dendritic cells were harvested on day 7.

rAAV Production and Titration. rAAV vectors were produced using a standard triple transfection method (Sena-Esteves and Gao, Cold Spring Harb Protoc; doi:10.1101/pdb.top095513, 2020). All serotypes evaluated encode the same GFP transgene. Viruses were purified by cesium chloride ultracentrifugation and titrated using both silver staining and quantitative polymerase chain reaction (qPCR).

AAV, KT 474 treatments and cytokine measurement Human monocytic dendritic cells were plated in 96 well format and treated with 1e5 MOI of AAV and KT 474 at doses indicated and harvested 24 hours later for downstream analysis such as cell cytotoxicity and target gene expression via flow cytometry. The media was collected for cytokine analysis. The cells were centrifugated at 2,000 rpm for 5 minutes, and the supernatants were collected to analyze cytokines. Cytokines were measured using luminex using the MILLIPLEX® Human Cytokine/Chemokine/Growth Factor Panel A 38 Plex Premixed Magnetic Bead Panel-Immunology Multiplex Assay Catalog #HCYTA-60K-PX38 following manufacturer's protocol.

Cell viability assessment and IRAK4 expression: Dendritic cells (DCs) were resuspended in the FACS buffer and transferred to a U-bottom 96-well plate. The cells were centrifuged at 2,000 rpm for 5 minutes, and the supernatant was discarded. To analyze the cell viability, Live/Dead negative staining cells were gated, and the percentage was determined to be the cell viability. LIVE/DEAD™ Fixable Near-IR Dead Cell Stain Kit, for 633 or 635 nm excitation Catalog #L34975 from Invitrogen was used for cell staining. For IRAK4 staining, dendritic cells were lysed and fixed with 1×BD Phosphoflow™ Lyse/Fix Buffer for 10 minutes at 37° C. Then, dendritic cells were permeabilized with BD Phosphoflow™ Perm Buffer III for 30 minutes at 4° C. Then, dendritic cells were stained with PE mouse anti-human IRAK4 antibody for 30 minutes at 4° C., washed, and run for flow cytometry. PE mouse anti-human IRAK4 (BD Biosciences, Cat. No. 560303) BD Phosphoflow™ Lyse/Fix Buffer (Cat. No. 558049) BD Phosphoflow™ Perm Buffer III, Cat. No. 558050).

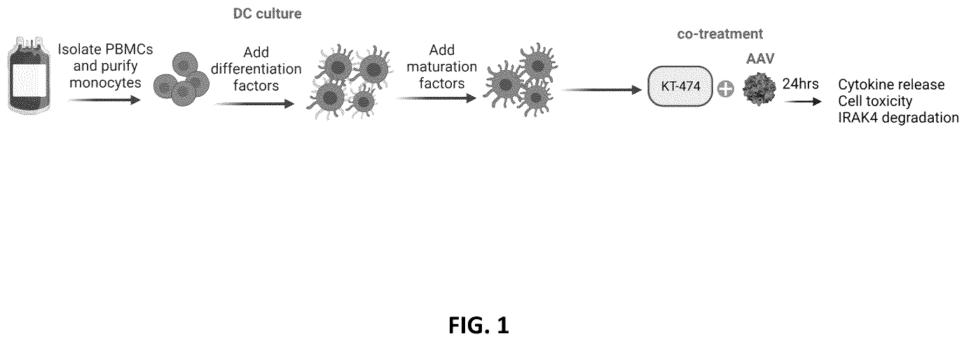

FIG. 1 shows the schema to assess effect of the IRAK4 degraders KT-474 on human dendritic cells treated with AAVs. In FIG. 1, peripheral blood monocuclear cells (PBMCs) were isolated from leukopaks. CD14+ monocytes were purified from (PBMCs), differentiaion factor cocktail is added to the monocytes to allow differentiation to dendritic cells and finally maturation factors were added to obtain mature dendritic cells. The mature dendritic cells were treated with KT-474 and AAV together (co-treatment) 24 hours post treatment. The media supernatant was collected for downstream analysis such as cytokine release. The cells were used to measure the level of IRAK4 degradation and assess cell viability.

### Results

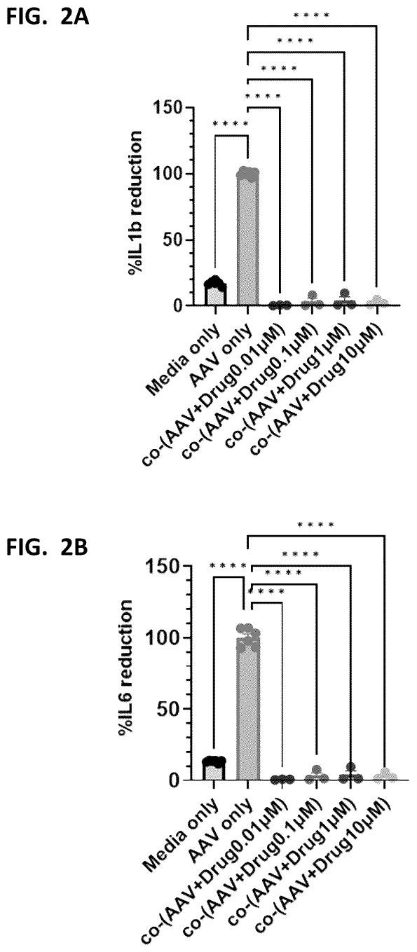

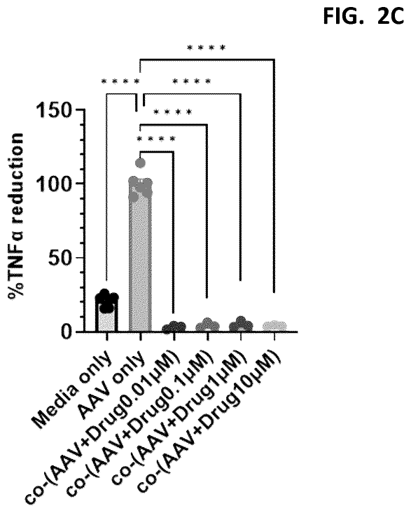

Human monocytic dendritic cells were co-treated with different doses of KT-474 as indicated and the same cells were infected with AAV at the same time at MOI of 1e5 for 24 hours and media supernatants were analysed for cytokine release. FIG. 2. Shows that co-treatment with KT-474 with AAV results in dampening of cytokine release Treatment with AAV induces secretion of IL1b (FIG. 2A), IL6 (FIG. 2B) and TNFa (FIG. 2C) cytokines from dendritic cells and this secretion is blocked by KT-474 at all doses. Each dot on the bar graph represents technical repeats of a representative donor. The experiment was performed on three donors and one way ANOVA was utilized to determine statistical significance. Cytokine secretion is expressed as a percentage across all donors.

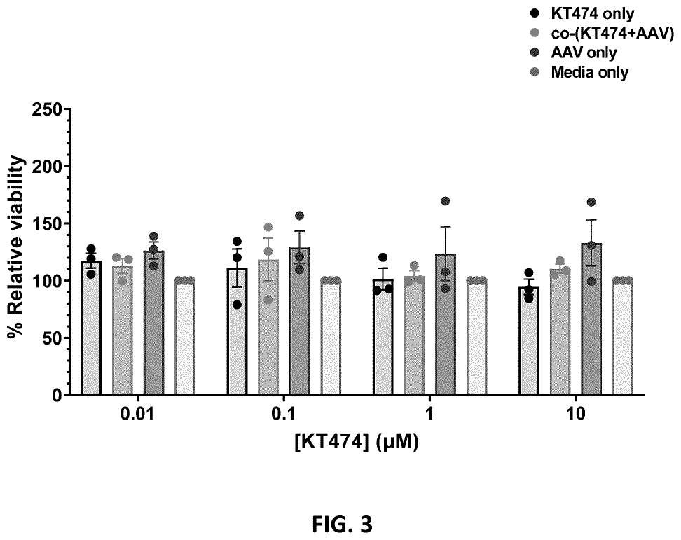

Human monocytic dendritic cells were treated with different doses of drugs as indicated and the same cells were infected with AAV at MOI of 1e5 for 24 hours. No significant difference was observed between the control (untreated/uninfected cells) as compared to the treated (AAV and KT474 co treated) and AAV infected cells indicating that KT-474 had no cellular toxicity at doses tested (FIG. 3). Each dot on bar graph represents a donor, the experiment was performed on three donors and one way ANOVA was utilized to determine statistical significance. Accordingly, treatment with KT-474 causes no cellular toxicity in primary human monocytic dendritic cells.

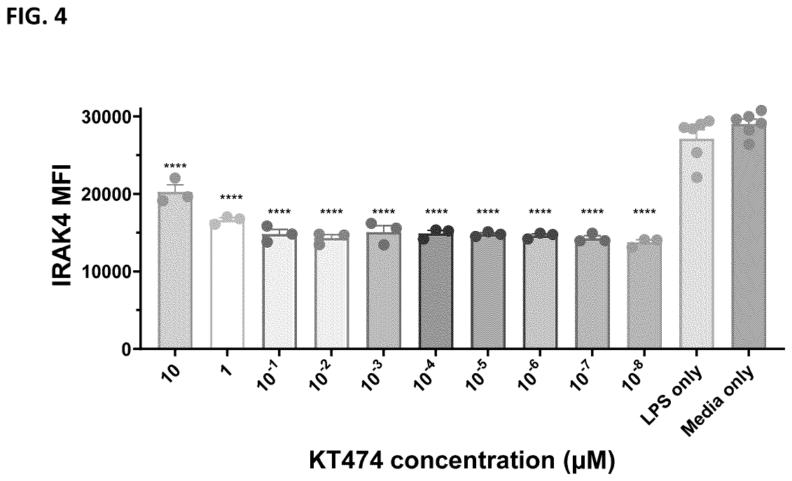

Human monocytic dendritic cells were treated with different doses of KT-474 as indicated and the same cells were infected with AAV at MOI of 1e5 for 24 hours. Cells were analysed for IRAK4 degradation using flow cytometry. KT-474 causes reduction of IRAK4 levels at all doses (FIG. 4). Each dot on bar graph represents technical repeats of a representative donor, the experiment was performed on three donors and one way ANOVA was utilized to determine statistical significance.

### Example 4: Analysis of the Effect of IRAK4 Degrader KT-474 on the Immune Response Induced by AAV in an In Vivo Mouse Model

### Materials and Methods

In vivo mice experiments: C57BL/6J mice were purchased from Jackson Laboratories. 6-8-week male mice were divided into 3 groups—PBS group, AAV group and AAV+KT474 group all groups with 6 mice each. The Compound of formula (I) (KT-474) was mixed with mouse chow Catalog #2016, Teklad Global 16% Protein Rodent Diets https://www.inotivco.com/rodent-natural-ingredient-2016-diets at a dose of 100 mg/kg. AAV+KT-474 group was fed the chow with KT474 15 days prior to AAV injection whereas PBS and AAV group were fed with mouse chow Catalog #2016, Teklad Global 16% Protein Rodent Diets https://www.inotivco.com/rodent-natural-ingredient-2016-diets. PBS group mice were injected intramuscularly with 100 ul PBS, AAV group was injected intramuscularly with 1e11 vg per quadricep muscle of both legs, 100 ul volume. AAV capsid used in the study had LacZ transgene. All animal experiments were conducted in accordance with the Institutional Animal Care and Use Committees at Sanofi, Framingham. Submandibular bleeds were performed weekly, and necropsy was performed three weeks post AAV injection.

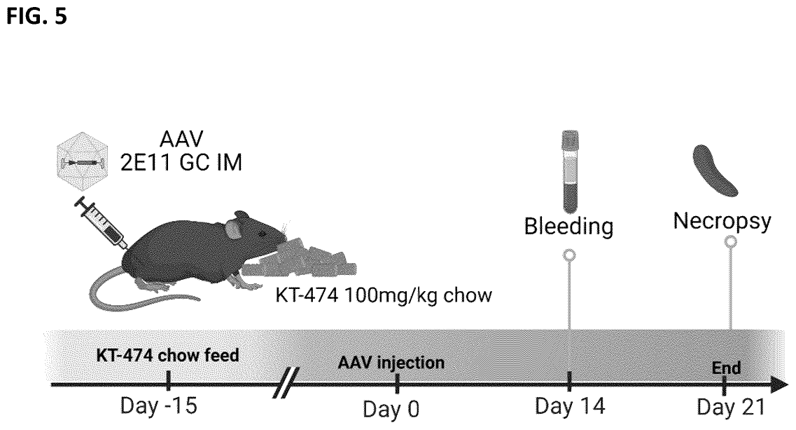

FIG. 5. shows the schematic of the in vivo mouse experiment. The mouse depicted shows group AAV+KT-474 group where KT-474 mixed chow was fed to the mice 15 days prior to AAV injection and then the blood was collected for PBMC analysis at day 14 and mouse necropsy was perfomed on day 21 to harvest spleen. The PBS and AAV only groups were fed regular chow and were bled on day 14 and necorpsied on day 21.

PBMC isolation 120 ul blood was collected in K2EDTA coated tubes. The blood was mixed with DBPS at 1:1 ration and pipetted into a separate tube containing Ficoll (GE17-5442-02) to ensure no mixing of the blood and Ficoll phases. The mixture was centrifuged at 2000 RPM for 25 minutes at room temperature (9 acceleration and no braking). The buffy coat containing the peripheral blood mononuclear cells (PBMCs) was collected, transferred to a new tube, and centrifuged for five minutes at 400 RCF. PBMCs were washed three times with phosphate buffered saline (PBS) containing 1% fetal bovine serum (FBS) or fetal calf serum (FCS) and counted. The PBMC cells thus obtained were stained with different antibodies to quantify percentage of different immune cell subsets in peripheral blood.

LacZ tetramer staining The PBMC was isolated from ˜120 μl of mouse blood, and it was transferred to a 96-well U-bottom plate. The PBMC was washed with the 200 μl of FACS buffer one time by centrifuging at 2,000 rpm for 5 minutes, and the PBMC was stained with a flow antibody cocktail (1:100 anti-CD4 PE-Cy7, 1:50 anti-CD8a FITC, 1:100 anti-CD62L APC, 1:100 anti-CD44 Pacific Blue, 1:100 Live/Dead Dead Cell Stain Kit, and 1:20 H-2Kb β-galactosidase tetramer) for 30 minutes at 4° C. After the incubation, the cells were washed with the FACS buffer 2 times and then fixed with 100 μl of BD Cytofix/Cytoperm Fixation/Permeabilization Solution Kit for 15 minutes at 4° C. The cells were washed with the FACS buffer 2 times, and then the samples were run on a flow cytometer (Novocyte Penteon Flow Cytometer Systems 5 Lasers, Agilent Technology).

### Results

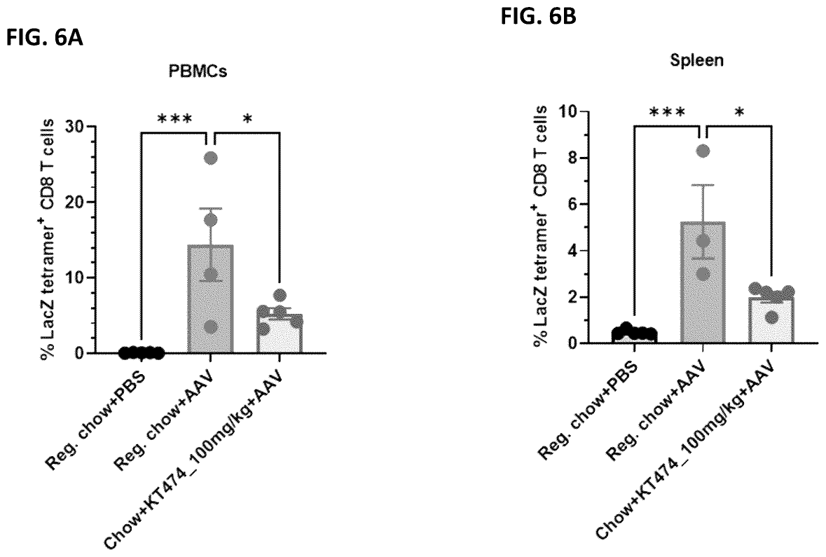

Mice were bled on day 14 post AAV injections and PBMCs were isolated. Splenocytes were harvest day 21 post AAV injection. PBMCs and splenocytes were stained with different antibodies to quantify for LacZ tetramer positive cells. These cell populations were upregulated upon AAV administration as compared to PBS controls and then reduced significantly upon KT-474 treatment (FIG. 6). FIG. 6A shows reduction of transgene LacZ specific CD8 T cells in PBMCs derived from peripheral blood on 14 days post AAV injections. FIG. 6B shows reduction of transgene LacZ specific CD8 T cells in spleen on 21 days post AAV injections The ROUT outlier method and One-way ANOV method was used to determine statistical significance. The ROUT method identifies outliers from nonlinear regression.

### Example 5: In Vivo Mouse Model to Assess the Effect of Oral Administration on IRAK4 Degrader KT-474 on the Immune Response Induced by AAV

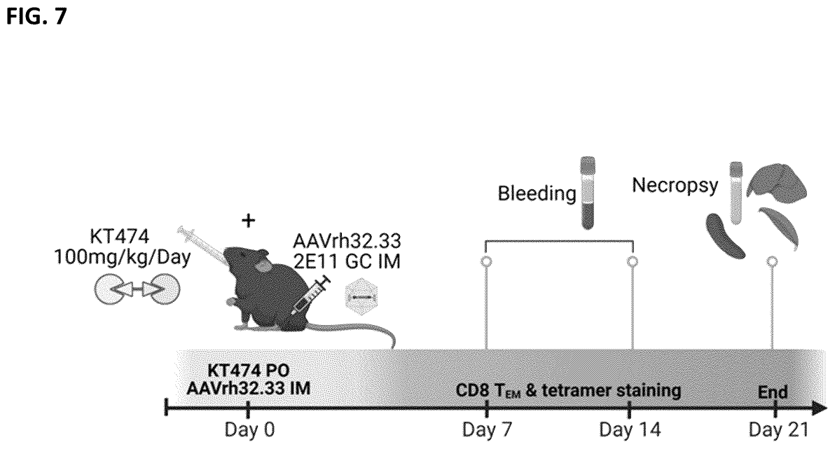

FIG. 7 shows a general schematic of an in vivo mouse model to assess the effect of oral administration on IRAK4 degrader KT-474 on the immune response induced by AAV. C57BL/6J mice are purchased from Jackson Laboratories. 6-8-week male mice are divided into 3 groups—PBS group, AAV group and AAV+KT474. KT-474 is being tested via oral route of administration treated twice daily at 100 mg/kg dose as a pre-treatment and co-treatment strategy. Oral administration of KT-474 is being tested in the clinic for other diseases https://clinicaltrials.gov/ct2/show/NCT04772885 and hence should be efficacious in blocking IRAK4 protein. Kymera therapeutics also shows evidence of complete degradation of IRAK4 protein using KT474 in human cells and in mouse tissues https://www.kymeratx.com/wp-content/uploads/2021/09/Euro-Prot-Deg-Summit-Sept-21-Final_Anthony-Slavin.pdf.

What is claimed is:

1. A method for delivering nucleic acid to a cell of an individual in need thereof, the method comprising
a) administering an IRAK degrader to the individual, and
b) administering a gene therapy agent to the individual.

2. A method for treating an individual in need thereof with a gene therapy agent, the method comprising
a) administering an IRAK degrader to the individual, and
b) administering the gene therapy agent to the individual.

3. A method for improving gene therapy in an individual in need thereof, the method comprising
a) administering an IRAK degrader to the individual, and
b) administering a gene therapy agent to the individual.

4. A method for suppressing an immune response to a gene therapy agent in an individual in need thereof, the method comprising
a) administering an IRAK degrader to the individual, and
b) administering a gene therapy agent to the individual.

5. The method of any one of claims 1-4 wherein the IRAK degrader modulates the activity or expression of an IRAK protein kinase.

6. The method of claim 5, wherein the IRAK protein kinase is an IRAK-1 protein kinase, an IRAK-2 protein kinase, an IRAK-3 protein kinase, or an IRAK-4 protein kinase.

7. The method of any one of claims 1-6, wherein the IRAK degrader modulates the activity or expression of an IRAK-4 protein kinase.

8. The method of any one of claims 1-7, wherein the IRAK degrader comprises the compound of formula [I]: 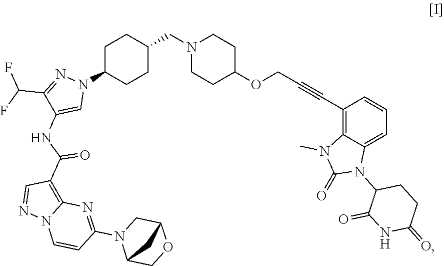 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21]
or a pharmaceutically acceptable salt thereof.

9. The method of any one of claims 1-8, wherein the gene therapy agent comprises a viral vector.

10. The method of claim 9, wherein the viral vector is an AAV particle.

11. The method of claim 10, wherein the AAV particle comprises an AAV1 capsid, an AAV2 capsid, an AAV3 capsid, an AAV4 capsid, an AAV5 capsid, an AAV6 capsid, an AAV7 capsid, an AAV8 capsid, an AAVrh8 capsid, an AAV9 capsid, an AAV10 capsid, an AAVrh10 capsid, an AAV11 capsid, an AAV12 capsid, an AAVrh32.33 capsid, An AAV-XL32 capsid, an AAV-XL32.1 capsid, an AAV LK03 capsid, an AAV2R471A capsid, an AAV2/2-7m8 capsid, an AAV DJ capsid, an AAV DJ8 capsid, an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, a mouse AAV capsid rAAV2/HBoV1 (chimeric AAV/human bocavirus virus 1), an AAV2HBKO capsid, an AAVPHP.B capsid or an AAVPHP.eB capsid, or a functional variant thereof.

12. The method of claim 11, wherein the AAV capsid comprises a tyrosine mutation, a heparin binding mutation, or an HBKO mutation.

13. The method of any one of claims 10-12, wherein the AAV viral particle comprises an AAV genome comprising one or more inverted terminal repeats (ITRs), wherein the one or more ITRs is an AAV1 ITR, an AAV2 ITR, an AAV3 ITR, an AAV4 ITR, an AAV5 ITR, an AAV6 ITR, an AAV7 ITR, an AAV8 ITR, an AAVrh8 ITR, an AAV9 ITR, an AAV10 ITR, an AAVrh10 ITR, an AAV11 ITR, or an AAV12 ITR.

14. The method of claim 13, wherein the one or more ITRs and the capsid of the AAV particle are derived from the same AAV serotype.

15. The method of claim 13, wherein the one or more ITRs and the capsid of the AAV particles are derived from different AAV serotypes.

16. The method of claim 9, wherein the viral vector is an adenoviral particle.

17. The method of claim 16, wherein the adenoviral particle comprises a capsid from Adenovirus serotype 2, 1, 5, 6, 19, 3, 11, 7, 14, 16, 21, 12, 18, 31, 8, 9, 10, 13, 15, 17, 19, 20, 22, 23, 24-30, 37, 40, 41, AdHu2, AdHu 3, AdHu4, AdHu24, AdHu26, AdHu34, AdHu35, AdHu36, AdHu37, AdHu41, AdHu48, AdHu49, AdHu50, AdC6, AdC7, AdC69, bovine Ad type 3, canine Ad type 2, ovine Ad, or porcine Ad type 3, or a functional variant thereof.

18. The method of claim 9, where the viral vector is a lentiviral particle.

19. The method of claim 18, wherein the recombinant lentiviral particle is pseudotyped with vesicular stomatitis virus (VSV), lymphocytic choriomeningitis virus (LCMV), Ross river virus (RRV), Ebola virus, Marburg virus, Mokala virus, Rabies virus, RD114, or a functional variant thereof.

20. The method of claim 9, where the viral vector is a Herpes simplex virus (HSV) particle.

21. The method of claim 20, wherein the HSV particle is an HSV-1 particle or an HSV-2 particle, or a functional variant thereof.

22. The method of any one of claims 1-8, wherein the gene therapy agent comprises a lipid nanoparticle.

23. The method of any one of claims 1-22, wherein the gene therapy agent comprises nucleic acid encoding a heterologous transgene.

24. The method of claim 23, wherein the heterologous transgene is operably linked to a promoter.

25. The method of claim 24, wherein the promoter is a constitutive promoter, a tissue-specific promoter, or an inducible promoter.

26. The method of any one of claims 1-25, wherein the IRAK degrader is administered before, at the same time, or after administration of the gene therapy agent.

27. The method of any one of claims 1-26, wherein the individual has a disease or disorder suitable for treatment by gene therapy.

28. The method of claim 27, wherein the disease or disorder is a monogenic disease or disorder.

29. The method of any one of claims 1-28, wherein the gene therapy agent is administered intravenously, intraperitoneally, intra-arterially, intramuscularly, subcutaneously, or intrahepatically.

30. The method of any one of claims 1-29, wherein the IRAK degrader is administered orally, intravenously, intraperitoneally, intra-arterially, intramuscularly, subcutaneously, or intrahepatically.

31. A method for delivering a nucleic acid to a cell of an individual in an individual in need thereof, the method comprising
a) incubating innate immune cells from the individual with the gene therapy agent,
b) analyzing the innate immune cells for the expression of one or more cytokines wherein expression of a cytokine signature following incubation with the gene therapy agent identifies an individual with innate immunity to the gene therapy agent,
c) administering an IRAK degrader to the individual identified in step b), and
d) administering the gene therapy agent to the individual identified in step b).

32. A method for treating an individual in need thereof, the method comprising
a) incubating innate immune cells from the individual with the gene therapy agent,
b) analyzing the innate immune cells for the expression of one or more cytokines wherein expression of a cytokine signature following incubation with the gene therapy agent identifies an individual with innate immunity to the gene therapy agent,
c) administering an IRAK degrader to the individual identified in step b), and
d) administering the gene therapy agent to the individual identified in step b).

33. A method for selecting an individual for treatment with a gene therapy agent and an IRAK degrader, the method comprising
a) incubating innate immune cells from the individual with the gene therapy agent,
b) analyzing the innate immune cells for the expression of one or more cytokines wherein expression of a cytokine signature following incubation with the gene therapy agent identifies an individual for treatment with a gene therapy agent and an IRAK degrader.
c) selecting the individual identified in step b) for treatment with a gene therapy agent and an IRAK degrader.

34. The method of any one of claims 31-33, wherein the innate immune cell is a dendritic cell, a monocyte, a macrophage or a natural killer (NK) cell.

35. The method of any one of claims 31-34, wherein the innate immune cells are isolated from peripheral blood mononuclear cells from the individual.

36. The method of any one of claims 31-35, wherein the innate immune cell is a dendritic cell.

37. The method of claim 36, wherein the dendritic cell is derived from a monocyte of the individual.

38. The method of claim 37 further comprising
isolating monocytes from the individual and
incubating the monocytes in dendritic cell culture media to derive dendritic cells from the monocytes prior to incubating the dendritic cells with the gene therapy agent.

39. The method of claim 37 or 38, wherein the monocytes are CD14+ monocytes.

40. The method of any one of claims 37-39, wherein the monocytes are incubates with the dendritic cell culture media for about 5 to about 10 days or about 7 to about 8 days to derive dendritic cells from the monocytes.

41. The method of any one of claims 31-40, wherein the dendritic cells are replated prior to the incubation with the gene therapy agent of step c).

42. The method of claim 41, wherein the dendritic cells are replated into microwell dishes.

43. The method of any one of claims 31-42, wherein the gene therapy agent is a viral vector, and wherein the innate immune cells are incubated with the viral vector at an MOI of about 1×10³ to about 1×10⁵ or about 1×10⁴.

44. The method of any one of claims 31-42, wherein the gene therapy agent is a non-viral vector, and wherein the innate immune cells are incubated with the non-viral vector at a concentration of about 1 ng/mL to about 1 mg/mL.

45. The method of any one of claims 31-44, wherein the innate immune cells are incubated with the gene therapy agent for about 12 hours to about 36 hours or about 24 hours.

46. The method of any one of claims 31-45, wherein the cytokine signature comprises increased expression of one or more of IL6, TNFα, IL-1β, MCP1 and MIP-1α.

47. The method of any one of claims 31-46, wherein the cytokine signature comprises increased expression of IL6, TNFα, IL-1β, MCP1 and MIP-1α.

48. The method of any one of claims 31-47, wherein the cytokine signature comprises increased expression of IL6, TNFα, and IL-1β.

49. The method of any one of claims 31-48, wherein expression of the cytokines in the cytokine signature is increased compared to a suitable control.

50. The method of claim 49, wherein the suitable control is the expression of the cytokines in the cytokine signature from innate immune cells that are not incubated with the gene therapy agent or wherein the suitable control is expression of the cytokines in the cytokine signature from innate immune cells prior to incubation with the gene therapy agent.

51. The method of any one of claims 31-50, wherein the IRAK degrader modulates the activity of an IRAK protein kinase.

52. The method of claim 51, wherein the IRAK protein kinase is an IRAK-1 protein kinase, an IRAK-2 protein kinase, an IRAK-3 protein kinase, or an IRAK-4 protein kinase.

53. The method of any one of claims 31-52, wherein the IRAK degrader modulates the activity of an IRAK-4 protein kinase.

54. The method of any one of claims 31-52, wherein the IRAK degrader is a small molecule.

55. The method of any one of claims 31-54, wherein the IRAK degrader comprises the compound of formula [I]: 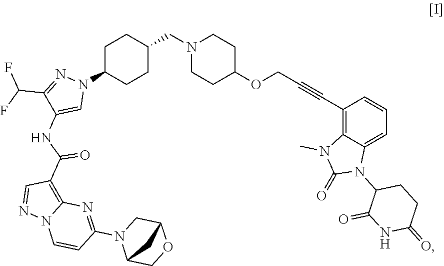 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21]
or a pharmaceutically acceptable salt thereof.

56. The method of any one of claims 31-55, wherein the IRAK degrader blocks TLR9 function.

57. The method of any one of claims 31-56, wherein the gene therapy agent is a viral vector.

58. The method of claim 57, wherein the viral vector is an AAV particle.

59. The method of claim 58, wherein the AAV particle comprises an AAV1 capsid, an AAV2 capsid, an AAV3 capsid, an AAV4 capsid, an AAV5 capsid, an AAV6 capsid, an AAV7 capsid, an AAV8 capsid, an AAVrh8 capsid, an AAV9 capsid, an AAV10 capsid, an AAVrh10 capsid, an AAV11 capsid, an AAV12 capsid, an AAVrh32.33 capsid, An AAV-XL32 capsid, an AAV-XL32.1 capsid, an AAV LK03 capsid, an AAV2R471A capsid, an AAV2/2-7m8 capsid, an AAV DJ capsid, an AAV DJ8 capsid, an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, a mouse AAV capsid rAAV2/HBoV1 (chimeric AAV/human bocavirus virus 1), an AAV2HBKO capsid, an AAVPHP.B capsid or an AAVPHP.eB capsid, or a functional variant thereof.

60. The method of claim 59, wherein the AAV capsid comprises a tyrosine mutation, a heparin binding mutation, or an HBKO mutation.

61. The method of any one of claims 58-60, wherein the AAV viral particle comprises an AAV genome comprising one or more inverted terminal repeats (ITRs), wherein the one or more ITRs is an AAV1 ITR, an AAV2 ITR, an AAV3 ITR, an AAV4 ITR, an AAV5 ITR, an AAV6 ITR, an AAV7 ITR, an AAV8 ITR, an AAVrh8 ITR, an AAV9 ITR, an AAV10 ITR, an AAVrh10 ITR, an AAV11 ITR, or an AAV12 ITR.

62. The method of claim 61, wherein the one or more ITRs and the capsid of the AAV particle are derived from the same AAV serotype.

63. The method of claim 61, wherein the one or more ITRs and the capsid of the AAV particles are derived from different AAV serotypes.

64. The method of claim 57, wherein the viral vector is an adenoviral particle.

65. The method of claim 64, wherein the adenoviral particle comprises an capsid from Adenovirus serotype 2, 1, 5, 6, 19, 3, 11, 7, 14, 16, 21, 12, 18, 31, 8, 9, 10, 13, 15, 17, 19, 20, 22, 23, 24-30, 37, 40, 41, AdHu2, AdHu 3, AdHu4, AdHu24, AdHu26, AdHu34, AdHu35, AdHu36, AdHu37, AdHu41, AdHu48, AdHu49, AdHu50, AdC6, AdC7, AdC69, bovine Ad type 3, canine Ad type 2, ovine Ad, or porcine Ad type 3, or a functional variant thereof.

66. The method of claim 57, where the viral vector is a lentiviral particle.

67. The method of claim 66, wherein the recombinant lentiviral particle is pseudotyped with vesicular stomatitis virus (VSV), lymphocytic choriomeningitis virus (LCMV), Ross river virus (RRV), Ebola virus, Marburg virus, Mokala virus, Rabies virus, RD114, or a functional variant thereof.

68. The method of claim 57, where the viral vector is a Herpes simplex virus (HSV) particle.

69. The method of claim 68, wherein the HSV particle is an HSV-1 particle or an HSV-2 particle, or a functional variant thereof.

70. The method of any one of claims 31-56, wherein the gene therapy agent is a lipid nanoparticle.

71. The method of any one of claims 31-70, wherein the gene therapy agent comprises nucleic acid encoding a heterologous transgene.

72. The method of claim 71, wherein the heterologous transgene is operably linked to a promoter.

73. The method of claim 72, wherein the promoter is a constitutive promoter, a tissue-specific promoter, or an inducible promoter.

74. The method of any one of claims 31-73, wherein the IRAK degrader is administered before, at the same time, or after administration of the gene therapy agent.

75. The method of any one of claims 31-74, wherein the individual has a disease or disorder suitable for treatment by gene therapy.

76. The method of claim 75, wherein the disease or disorder is a monogenic disease or disorder.

77. The method of any one of claims 31-76, wherein the gene therapy agent is administered intravenously, intraperitoneally, intra-arterially, intramuscularly, subcutaneously, or intrahepatically.

78. The method of any one of claims 31-77, wherein the IRAK degrader is administered orally, intravenously, intraperitoneally, intra-arterially, intramuscularly, subcutaneously, or intrahepatically.

79. The method of any one of claims 1-78, wherein the IRAK modulator activates CD8 T cells.

80. Use of a composition in the manufacture of a medicament for delivering nucleic acid to a cell of an individual in need thereof, wherein the composition comprises a gene therapy agent, and wherein the composition is formulated for use in combination with an IRAK degrader.

81. Use of a composition in the manufacture of a medicament for delivering nucleic acid to a cell of an individual in need thereof, wherein the composition comprises an IRAK degrader, and wherein the composition is formulated for use in combination with a gene therapy agent.

82. Use of a composition in the manufacture of a medicament for treating an individual in need of gene therapy, wherein the composition comprises a gene therapy agent, and wherein the composition is formulated for use in combination with an IRAK degrader.

83. Use of a composition in the manufacture of a medicament for treating an individual in need of gene therapy, wherein the composition comprises an IRAK degrader, and wherein the composition is formulated for use in combination with a gene therapy agent.

84. Use of a composition in the manufacture of a medicament for modulating an immune response to gene therapy in an individual in need of gene therapy, wherein the composition comprises a gene therapy agent, and wherein the composition is formulated for use in combination with an IRAK degrader.

85. Use of a composition in the manufacture of a medicament for modulating an immune response to gene therapy in an individual, wherein the composition comprises an IRAK degrader, and wherein the composition is formulated for use in combination with a gene therapy agent.

86. The use of any one of claims 80-85, wherein the gene therapy agent is an AAV particle, an adenovirus particle, a lentivirus particle, a HSV particle, or a lipid nanoparticle.

87. The use of any one of claims 80-86, wherein the IRAK degrader is an IRAK-4 degrader.

88. A composition comprising a gene therapy agent for use in delivering nucleic acid to a cell of an individual in need thereof, wherein the gene therapy agent is used in combination with an IRAK degrader.

89. A composition comprising an IRAK degrader for use in delivering nucleic acid to a cell of an individual in need thereof, wherein the IRAK degrader is used in combination with a gene therapy agent.

90. A composition comprising a gene therapy agent for use in treating an individual in need of gene therapy, wherein the gene therapy agent is used in combination with an IRAK degrader.

91. A composition comprising an IRAK degrader for use in treating an individual in need of gene therapy, wherein the IRAK degrader is used in combination with a gene therapy agent.

92. A composition comprising an IRAK degrader for modulating an immune response to gene therapy in an individual in need of gene therapy, wherein the IRAK degrader is used in combination with a gene therapy agent.

93. A composition comprising an IRAK degrader for suppressing an immune response to gene therapy in an individual in need of gene therapy, wherein the IRAK degrader is used in combination with a gene therapy agent.

94. The composition of any one of claims 88-93, wherein the gene therapy agent is an AAV particle, an adenovirus particle, a lentivirus particle, a HSV particle, or a lipid nanoparticle.

95. The composition of any one of claims 88-94, wherein the IRAK degrader is an IRAK-4 degrader.

96. A kit for use in the method of any one of claims 1-79.

97. A kit for the use of any one of claims 80-87.

98. A kit comprising the composition of any one of claims 92-97.
